(19) United States
(12) Patent Application Publication
(10) Pub. No.: US 2022/0183621 A1
(43) Pub. Date: Jun. 16, 2022

(54) PREDICTING CORE PHENOTYPING DOMAINS OF LOW BACK PAIN WITH MULTIMODAL BRAIN IMAGING METRICS

(71) Applicant: Washington University, St. Louis, MO (US)

(72) Inventors: Ammar Hawasli, St. Louis, MO (US); Bidhan Lamichhane, St. Louis, MO (US); Dinal Jayasekera, St. Louis, MO (US); Eric Leuthardt, St. Louis, MO (US); Wilson Ray, St. Louis, MO (US)

(73) Assignee: Washington University, St. Louis, MO (US)

(21) Appl. No.: 17/553,394

(22) Filed: Dec. 16, 2021

Related U.S. Application Data
(60) Provisional application No. 63/126,199, filed on Dec. 16, 2020.

(51) Int. Cl. A61B 5/00; A61B 5/055; G16H 50/30

(52) U.S. Cl. CPC A61B 5/4824; A61B 5/7275; A61B 5/055; G16H 50/30; A61B 5/4064; A61B 5/7267; A61B 5/0042

(57) ABSTRACT

A multi-modal biomarker predictive of a pain level in a patient that includes at least one of a structural MRI-based parameter and a functional MRI-based parameter from the brain of the patient is described. Systems and computer-implemented methods of estimating a pain level in a patient based that transform the multimodal into the estimated pain level using a machine learning model are also disclosed.

### CROSS-REFERENCE TO RELATED APPLICATIONS

This application claims the benefit of priority to U.S. Provisional Application Ser. No. 63/126,199 filed on Dec. 16, 2020, the content of which is incorporated herein by reference in its entirety.

### FIELD OF THE DISCLOSURE

The present disclosure generally relates to multimodal biomarkers that include structural and functional MRI-related and related methods for predicting patient-reported outcome scores, disability, and pain scores for low back pain.

### BACKGROUND OF THE DISCLOSURE

Back pain is a disorder with a relatively high prevalence and high health care costs associated with treatment. A common cause of back pain is myelopathy, a disorder resulting from severe compression of the spinal cord. Causes of myelopathy include spinal stenosis, spinal trauma, and spinal infections, as well as autoimmune, oncological, neurological, and congenital disorders. Radiculopathy, the pinching of the nerve roots as they exit the spinal cord or cross the intervertebral disc, may accompany myelopathy. Myelopathy may occur at any location along the spinal cord including cervical, thoracic, and in rare cases lumbar regions, although cervical myopathy is most prevalent.

Cervical myelopathy (CM) is a common form of spinal cord injury with a poorly defined natural history. CM is typically characterized as a progressive and chronic compression injury to the spinal cord. CM is typically treated using surgery, but surgical outcomes are variable with about 33% of patients improving, about 40% of patients remaining stable, and about 25% of patients worsening.

Chronic low back pain (LBP) is a major cause of disability for many individuals globally. LBP is three times more likely to develop in individuals over the age of 50 when compared to individuals under 30. In the United States, LBP is linked to higher healthcare costs and reduced productivity with total costs estimated at $100 billion in 2006. Although spinal MRI techniques are actively utilized in the investigation of biomarkers for LBP, many individuals with LBP show no significant abnormalities in modern spinal imaging. These hurdles and the complex pathophysiology of chronic LBP make its prognostication and clinical management challenging. There is a clinical need for easily accessible noninvasive biomarkers that, when met, would facilitate early diagnoses leading to earlier treatment plans with improved outcomes and would further understanding of disease progression and severity.

Chronic low back pain (LBP) is a very common health problem worldwide and a major cause of disability. Yet, the lack of quantifiable metrics on which to base clinical decisions leads to imprecise treatments, unnecessary surgery, and reduced patient outcomes. Although the focus of LBP has largely focused on the spine, the literature demonstrates a robust reorganization of the human brain in the setting of LBP. Brain neuroimaging holds promise for the discovery of biomarkers that will improve the treatment of chronic LBP.

Chronic low back pain (LBP) represents a significant public health problem and is a major cause of disability globally. Health care costs for LBP in the United States have ballooned to nearly $1 trillion. The diagnosis and treatment of chronic LBP have been complicated by heterogeneous etiologies and neuroimaging modalities that fail to measure central mechanisms of pain. Spinal magnetic resonance imaging (MRI) techniques are actively utilized in the investigation of biomarkers of LBP but are often limited by artifacts imposed by spinal implants necessary for stabilization and also do not measure central pain processing mechanisms. It is well known that many individuals with LBP show no significant abnormalities in modern spinal imaging. These hurdles and the complex pathophysiology of chronic LBP make its prognostication and clinical management challenging.

Brain imaging has identified regions that are involved in the processing and perception of pain. The cortical areas identified are involved in motor processing (primary motor cortex, supplementary motor area), multisensory integration (temporal-parietal junction), cognitive perception of pain (anterior cingulate cortex, ventromedial prefrontal cortex, dorsolateral prefrontal cortex), and act as nociceptive centers of pain (insula, thalamus). However, neural correlates of LBP remain poorly understood. Cortical thickness (CT) appears to reflect the functional organization of the human cortex and acts as a potential marker for the development of LBP. Regional changes in grey matter have been reported in several pain studies. A global reduction in grey matter volume and a disruption of the whole-brain morphological organization has been previously demonstrated in LBP patients. Subjects who clinically recovered typically had normal gray matter volumes, but subjects with persistent LBP demonstrated global and regional reductions in gray matter volume.

Resting-state functional connectivity (rsFC) is commonly used as a noninvasive biomarker for various neurological conditions including Alzheimer's disease, and Parkinson's disease. Resting-state functional MRI (rsfMRI) has gained some popularity in measuring functional connectivity between brain regions and resting-state networks (RSN) in patients with LBP. Experiments have reported disruptions in connectivity within the visual processing stream and between the insula and pain processing areas of LBP patients. Similar observations have been reported on connectivity between the nucleus accumbens and the medial prefrontal cortex. Furthermore, there is increasing evidence from other neurological disorders that damage to one part of the central nervous system (CNS) can disrupt connectivity patterns within other CNS structures. This can lead to disturbances in network connectivity on a global brain level. However, previous studies lack a systematic analysis of global patterns of rsFC, and the brain's intra- and inter-network interactions in LBP.

Very few studies have investigated rsFC in clinical practice as a biomarker for LBP. Several putative non-imaging biomarkers have been investigated in LBP, but these biomarkers are often invasive and do not assess the impact of LBP on the brain. There is increasing evidence from other neurological disorders that damage to one part of the central nervous system can lead to disturbances in network connectivity on a global brain level. Changes have been reported in the network organization of individuals with chronic pain disorders including LBP. Graph theory measures can be used to model patterns of resting-state connectivity consisting of nodes (cortical areas) and edges (functional interactions between brain regions).

Patterns of resting-state connectivity can also be modeled using graph theoretical measures consisting of nodes (brain parcels) and edges (functional interactions between brain regions). The organization of these RSNs is critical to the flow of information between nodes and its resulting efficiency. Hubs play a key role in facilitating more efficient integration of information between nodes by adopting a highly connected and functionally central role within a network. Changes have been reported in the network organization of individuals with chronic pain disorders and LBP. However, these studies did not examine hubs specifically. Instead, they assessed the variability in node community membership. The highly-connected nature of hubs creates an inherent vulnerability in the event of a disruption to its organization. This can result in a significant interruption in the flow of information. Hubs are disproportionally affected in neurological disorders as changes in CT are more likely to occur in hubs.

Other objects and features of the disclosure will be in part apparent and in part pointed out hereinafter.

### SUMMARY

In various aspects, multi-modal biomarkers and methods of estimating a pain level of a patient based on the multi-modal biomarkers are disclosed herein.

In one aspect, a multi-modal biomarker predictive of a pain level in a patient is disclosed that includes at least one of a structural MRI-based parameter from a brain of the patient and a functional MRI-based parameter from the brain of the patient. In some aspects, the structural MRI-based parameter includes at least one of a cortical thickness and a sub-cortical volume and the functional MRI-based parameter includes at least one of a resting-state functional connectivity matrix parameter and a graph metric parameter; the graph parameter includes the global efficiency, the clustering coefficient, and the characteristic path length. In some aspects, the resting-state functional connectivity matrix parameter includes a global connectivity. In some aspects, the biomarker is cortical thickness, sub-cortical volume, and global connectivity.

In another aspect, a computer-implemented method of estimating a pain level in a patient based on a multi-modal biomarker is disclosed that includes providing to a computing device the multi-modal biomarker. The multimodal biomarker includes at least one of a structural MRI-based parameter from a brain of the patient and a functional MRI-based parameter from the brain of the patient. The method further includes transforming, using the computing device, the multi-modal biomarker into the estimated pain level using a machine learning model. In some aspects, the machine learning model includes a support vector machine. In some aspects, the structural MRI-based parameter includes at least one of a cortical thickness and a sub-cortical volume and the functional MRI-based parameter includes at least one of a resting-state functional connectivity matrix parameter and a graph metric parameter; the graph metric parameter includes at least one of the global efficiency, the clustering coefficient, and the characteristic path length. In some aspects, the resting-state functional connectivity matrix parameter includes global connectivity. In some aspects, the multi-modal biomarker is the cortical thickness, the sub-cortical volume, and the global connectivity. In some aspects, the estimated pain level includes an estimated clinical score that includes a score from at least one of a Modified Japanese Orthopedic Association, a Oswestry Disability Index, an SF-36, a Disabilities of Arm, Shoulder and Hand, a Neck Disability, a Rolland Morris Pain Questionnaire, a McGill Pain Questionnaire, a Shoulder Pain Score, any portion thereof, and any combination thereof. In some aspects, the method further includes training, using the computing device, the machine learning model using a training dataset, where the training dataset includes a plurality of entries. Each entry includes a multimodal biomarker and an associated clinical score for a training patient from a population of pain patients.

A system for estimating a pain level in a patient based on a multi-modal biomarker, the system comprising a computing device comprising at least one processor and a non-volatile computer-readable media, the non-volatile computer-readable media containing instructions executable on the at least one processor to transform the multi-modal biomarker into the estimated pain level using a machine learning model. In some aspects, the machine learning model includes a support vector machine. In some aspects, the structural MRI-based parameter includes at least one of a cortical thickness and a sub-cortical volume and the functional MRI-based parameter includes at least one of a resting-state functional connectivity matrix parameter and a graph metric parameter; the graph metric parameter includes at least one of a global efficiency, a clustering coefficient, and a characteristic path length. In some aspects, the resting-state functional connectivity matrix parameter includes a global connectivity. In some aspects, the multi-modal biomarker is the cortical thickness, the sub-cortical volume, and the global connectivity. In some aspects, the estimated pain level includes an estimated clinical score from at least one of a Modified Japanese Orthopedic Association, a Oswestry Disability Index, an SF-36, a Disabilities of Arm, Shoulder and Hand, a Neck Disability, a Rolland Morris Pain Questionnaire, a McGill Pain Questionnaire, a Shoulder Pain Score, any portion thereof, and any combination thereof. In some aspects, the non-volatile computer-readable media further contains instructions executable on the at least one processor to train the machine learning model using a training dataset that includes a plurality of entries, each entry comprising a multimodal biomarker and an associated clinical score for a training patient from a population of pain patients.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow chart representing a data processing pipeline for the development of SVM classification models based on graph theory features of resting-state functional connectivity (rsFC) matrices.

FIG. 2 contains a series of cortical maps summarizing frequently selected features used in the process of developing SVM classifier models as summarized in FIG. 1. The frequency of selection for each cortical feature used to train the SVM model using BC+CC+DC and Enet-subset feature selection method was plotted onto a cortical mesh surface. The top 60 features were selected in all 100 iterations and sorted according to the frequency of their selection during the 100 iterations. Cortical areas outlined in green are bilateral while those outlined in black are not.

FIG. 3 are cortical maps summarizing the bilateral frequently selected features used in the process of developing SVM classifier models as summarized in FIG. 1. Bilateral cortical regions from the 60 most frequently selected parcels used to train the SVM model using BC+CC+DC and an Enet-subset feature selection method are highlighted on a cortical mesh surface of the left hemisphere showing the frequency of selection. Cortical areas are outlined in green and labeled accordingly.

FIG. 4A is a graph comparing the betweenness centrality (BC) distributions of a control group and a group of patients with low back pain.

FIG. 4B is a graph comparing the clustering coefficient (CC) distributions of a control group and a group of patients with low back pain.

FIG. 4C is a graph comparing the degree centrality (DC) distributions of a control group and a group of patients with low back pain.

FIG. 4D is a graph comparing the local efficiency (LE) distributions of a control group and a group of patients with low back pain.

FIG. 5 contains cortical maps summarizing the frequency, as a color gradient, of each cortical area that contributed to the classification accuracy of the Enet-subset model when using betweenness centrality data only. The cortical areas outlined in black are the top 60 cortical areas that contributed to the classification accuracy of this model, ranked in descending order by frequency.

FIG. 6 is a block diagram schematically illustrating a system in accordance with one aspect of the disclosure.

FIG. 7 is a block diagram schematically illustrating a computing device in accordance with one aspect of the disclosure.

FIG. 8 is a block diagram schematically illustrating a remote or user computing device in accordance with one aspect of the disclosure.

FIG. 9 is a block diagram schematically illustrating a server system in accordance with one aspect of the disclosure.

FIG. 10 contains cortical maps summarizing the frequency, as a color gradient, of each cortical area that contributed to the classification accuracy of the Enet-subset model when using clustering coefficient data only. The cortical areas outlined in black are the top 60 cortical areas that contributed to the classification accuracy of this model, ranked in descending order by frequency.

FIG. 11 contains cortical maps summarizing the frequency, as a color gradient, of each cortical area that contributed to the classification accuracy of the Enet-subset model when using degree centrality data only. The cortical areas outlined in black are the top 60 cortical areas that contributed to the classification accuracy of this model, ranked in descending order by frequency.

FIG. 12 contains cortical maps summarizing the bilateral cortical regions from the 60 most frequently selected parcels used to train the SVM model using BC+CC+DC and an Enet-subset feature selection method, shown projected onto a cortical mesh surface of the right hemisphere. Cortical areas are outlined in green and labeled accordingly.

FIG. 13 contains cortical maps summarizing the consequences of chronic low back pain (LBP) on cortical thickness. The cortical map summarizes the weight (beta parameter) of group effect in CT. The positive beta (red) represents areas of thicker cortex in controls. The negative beta (blue) represents the cortex that is thicker in LBP compared to HC. The parcels outlined with a black boundary show significant group differences between HC and LBP (p<0.05, uncorrected), and those outlined in green show significant group differences that survive FDR correction (q<0.05).

FIG. 14A contains cortical maps summarizing the beta parameter of Physical Component Summary (PCS) factor score when predicting CT in a general linear model after correcting for age (age-adjusted regression analysis). The parcels outlined in black are significantly correlated (predicted) with clinical factor scores (p<0.05, uncorrected), and those outlined in green show significant group differences that survive FDR correction (q<0.05). Since a higher clinical score suggests a better health condition, a negative beta (blue regions) suggests a thinner cortex in the HC group compared with LBP (thicker cortex in LBP). Similarly, the positive beta (red regions) suggests thicker cortical regions in HC (thinner cortex in LBP patients).

FIG. 14B contains cortical maps similar to the maps of FIG. 14A summarizing the beta parameter of Mental Component Summary (MCS) factor score when predicting CT in a general linear model after correcting for age (age-adjusted regression analysis).

FIG. 15A contains a connectivity map summarizing group differences in rsFC (Fisher's Z transformed) between LBP and HC groups. Note that red regions represent cortical areas or networks with reduced rsFC in LBP when compared to HC and blue regions represent increases. The lower triangle shows the z-scores for differences in rsFC between cortical areas grouped by network. A color code was assigned to significant (p<0.05, uncorrected) differences.

FIG. 15B contains a graph summarizing the differences in average connectivity between each pair of resting-state networks in terms of z-values (*=p<0.05, uncorrected; **=p<0.001, uncorrected).

FIG. 15C contains cortical maps of resting-state networks as outlined by the Cole-Anticevic Brain Network Parcellation.

FIG. 16 contains cortical maps of the DMN network, which consists of the medial prefrontal cortex, posterior cingulate cortex, inferior parietal cortex, and precuneus.

FIG. 17A contains cortical maps summarizing graph-theory hubs that were common to LBP and HC.

FIG. 17B contains cortical maps summarizing graph-theory hubs that were common to HC but not to LBP patients.

FIG. 17C contains cortical maps summarizing graph-theory hubs that were common to LBP patients but not to HC.

FIG. 18A is a Receiver Operating Characteristic (ROC) curve plot for the LBP vs HC classification.

FIG. 18B contains cortical maps summarizing the frequency of parcels used to train the SVM model using cortical thickness. Parcels outlined in black are the top 40 most frequently contributing parcels to the classification of the patient group when using CT. Parcels outlined in green have the highest frequency (51, i.e., selected as features in all LOO iterations) when contributing to the classification of all patients.

FIG. 19 is a schematic flow chart illustrating a multimodal biomarker for spinal pain disorders, as well as potential methods of using the biomarker to select a treatment.

FIG. 20 contains cortical maps comparing the cortical thickness distribution of a control group and a group of patients with chronic back pain (red: CBP<CON, blue: CPB>CON).

FIG. 21 contains cortical maps comparing the myelin content distribution of a control group and a group of patients with chronic back pain (red: CBP<CON, blue: CPB>CON).

FIG. 22 contains a series of images comparing the grey matter volume distribution of a control group and a group of patients with chronic back pain within various brain regions (red: CBP<CON, blue: CPB>CON).

FIG. 23A contains an image (left) and graph (right) comparing subcortical grey matter volumes of CM (cervical myelopathy), LBP (low back pain), and healthy control (CON) groups within a left accumbens region. Bar graphs include p-values of paired T-tests.

FIG. 23B contains an image (left) and graph (right) comparing subcortical grey matter volumes of CM (cervical myelopathy), LBP (low back pain), and healthy control (CON) groups within a right accumbens region. Bar graphs include p-values of paired T-tests.

FIG. 23C contains an image (left) and graph (right) comparing subcortical grey matter volumes of CM (cervical myelopathy), LBP (low back pain), and healthy control (CON) groups within a left caudate region. Bar graphs include p-values of paired T-tests.

FIG. 23D contains an image (left) and graph (right) comparing subcortical grey matter volumes of CM (cervical myelopathy), LBP (low back pain), and healthy control (CON) groups within a right caudate region. Bar graphs include p-values of paired T-tests.

FIG. 23E contains an image (left) and graph (right) comparing subcortical grey matter volumes of CM (cervical myelopathy), LBP (low back pain), and healthy control (CON) groups within a brain stem region. Bar graphs include p-values of paired T-tests.

FIG. 23F contains an image (left) and graph (right) comparing subcortical grey matter volumes of CM (cervical myelopathy), LBP (low back pain), and healthy control (CON) groups within a left putamen region. Bar graphs include p-values of paired T-tests.

FIG. 23G contains an image (left) and graph (right) comparing subcortical grey matter volumes of CM (cervical myelopathy), LBP (low back pain), and healthy control (CON) groups within a right putamen region. Bar graphs include p-values of paired T-tests.

FIG. 23H contains an image (left) and graph (right) comparing subcortical grey matter volumes of CM (cervical myelopathy), LBP (low back pain), and healthy control (CON) groups within a left cerebellum region. Bar graphs include p-values of paired T-tests.

FIG. 23I contains an image (left) and graph (right) comparing subcortical grey matter volumes of CM (cervical myelopathy), LBP (low back pain), and healthy control (CON) groups within a right cerebellum region. Bar graphs include p-values of paired T-tests.

FIG. 23J contains an image (left) and graph (right) comparing subcortical grey matter volumes of CM (cervical myelopathy), LBP (low back pain), and healthy control (CON) groups within a left ventral diencephalon region. Bar graphs include p-values of paired T-tests.

FIG. 23K contains an image (left) and graph (right) comparing subcortical grey matter volumes of CM (cervical myelopathy), LBP (low back pain), and healthy control (CON) groups within a right ventral diencephalon region. Bar graphs include p-values of paired T-tests.

FIG. 24A is a heat map summarizing changes in whole-brain connectivity (above-diagonal) and cortex connectivity (below-diagonal) of the various resting-state networks in CPB relative to CON; red denotes connectivities where CPB<CON and blue denotes connectivities where CPB>CON.

FIG. 24B contains cortical maps of the changes in connectivity shown in FIG. 24A.

FIG. 25 contains cortical maps summarizing changes in functional connectivity to subgenual cingulate gyrusC in CPB relative to CON.

FIG. 26 is a map summarizing the correlations of global connectivity to a subset of the clinical scores of Table 14.

FIG. 27 is a graph summarizing correlations of efficiency of various resting-state networks to a subset of the clinical scores of Table 14.

FIG. 28 is a series of graphs summarizing SVM-predicted scores of a subset of the clinical tests of Table 14 as a function of the reported scores.

Those of skill in the art will understand that the drawings, described below, are for illustrative purposes only. The drawings are not intended to limit the scope of the present teachings in any way.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION OF THE DISCLOSURE

In various aspects, methods and algorithms to predict patient-reported outcome scores, disability, and pain scores for low back pain using brain imaging data including cortical thickness, surface area, sub-cortical volume, myelin content, and functional connectivity are disclosed herein.

In various aspects, brain imaging, including, but not limited to, structural and functional MRI, is used to predict pain scores and disability for low back pain. The methods disclosed herein not only prognosticate outcomes for low back pain but can be used to predict who should receive treatments and what types of treatments. The disclosed methods also identify regions of the brain that can be targeted to treat low back pain.

### I. Multi-Modal Pain Biomarker

In various aspects, a multi-modal biomarker predictive of a pain level in a patient is disclosed. The biomarker includes at least one of a structural MRI-based parameter and a functional MRI-based parameter derived from MRI signals obtained from the brain of the patient using an MRI scanner as described herein. In some aspects, the disclosed biomarker may include a structural MRI-based parameter including, but not limited to, a cortical thickness, a sub-cortical volume, and any other suitable structural MRI-based parameter without limitation. In other aspects, the disclosed biomarker may include a functional MRI-based parameter including, but not limited to, at least a portion of a resting state network functional connectivity matrix, parameters derived from a resting state network functional connectivity matrix such as global connectivity or a graph metric parameter, and any combination thereof. Non-limiting examples of suitable graph metric parameters include a global efficiency, a clustering coefficient, a characteristic path length, and any combination thereof.

In various aspects, the multimodal biomarker may be validated by comparing biomarkers obtained from a population of pain patients to corresponding biomarkers obtained from a healthy control population. In other aspects, threshold values or threshold ranges may be produced based on analysis of the biomarkers obtained from the healthy control population.

In additional aspects, the biomarker threshold values or ranges described above may be used to classify a patient with an unknown pain status based on a comparison of the patient's biomarker with the biomarker threshold values or ranges. In these additional aspects, the patient is classified as having pain if the patient's biomarker falls outside of the threshold values or ranges defined within the healthy control population. In more additional aspects, the comparison of a patient's biomarker to the threshold values or ranges may also be used to select a treatment.

Additional details are provided in the examples below.

### II. Machine Learning-Based Methods

In various aspects, machine learning-based methods of classifying a pain patient, predicting a pain level, and/or selecting a treatment for a patient based on the multi-mode biomarker described above are also disclosed herein. The disclosed machine-based methods include transforming the multi-mode biomarker into a patient classification, a predicted pain level, and/or treatment recommendation using a machine learning model. The machine learning model is trained using a training dataset that includes a plurality of entries. Each entry of the training set in one aspect includes a biomarker from a patient and a corresponding clinical pain score or other clinical pain measurements. Any suitable machine learning model may be used without limitation including, but not limited to, a support vector machine.

Additional details are provided in the examples below.

### III. Computing Systems and Devices

In various aspects, the methods described herein may be implemented using a computing device or computing system. Various non-limiting examples of suitable computing devices and systems are described below.

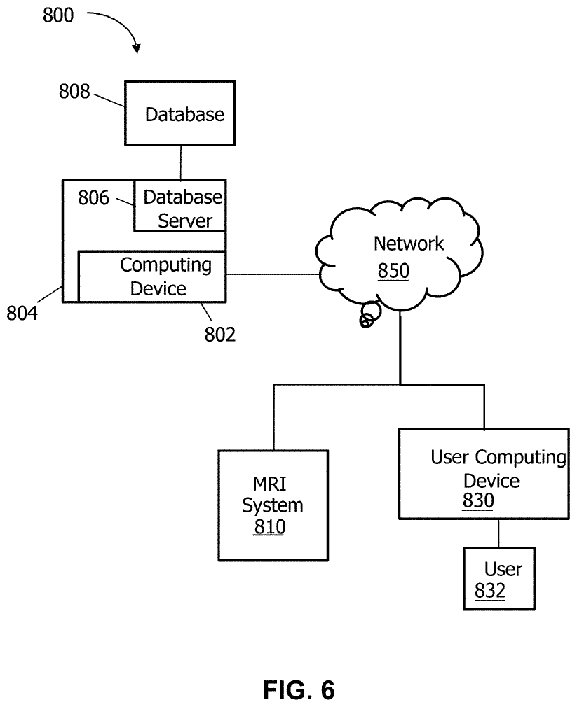

FIG. 6 depicts a simplified block diagram of a system 800 for implementing the methods described herein. As illustrated in FIG. 6, system 800 may be configured to implement at least a portion of the tasks associated with the disclosed method. The system 800 may include a computing device 802. In one aspect, the computing device 802 is part of a server system 804, which also includes a database server 806. The computing device 802 is in communication with a database 808 through the database server 806. The computing device 802 is communicably coupled to the MR imaging system 810 and a user computing device 830 through a network 850. The network 850 may be any network that allows local area or wide area communication between the devices. For example, the network 850 may allow the communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 830 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

In various other aspects, the computing device 802 is also communicably coupled to an MRI system 810 configured to obtain fMRI data and structural MRI data used to produce and interpret the biomarker using the methods described herein.

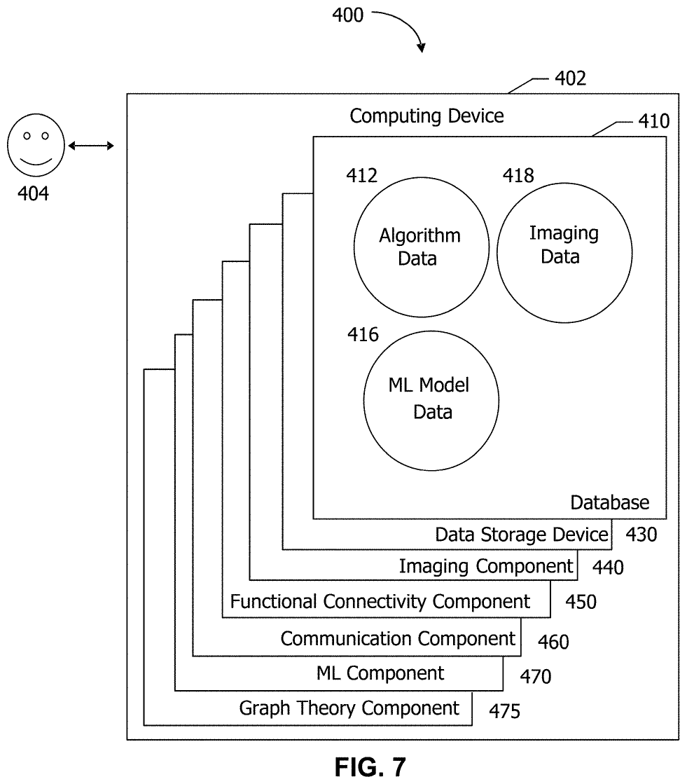

In other aspects, the computing device 802 is configured to perform a plurality of tasks associated with the method of calculating the biomarker based on functional connectivity as described herein. FIG. 7 depicts a component configuration 400 of a computing device 402, which includes a database 410 along with other related computing components. In some aspects, the computing device 402 is similar to computing device 802 shown in FIG. 6. A user 404 may access components of the computing device 402. In some aspects, database 410 is similar to database 808 shown in FIG. 6.

Referring again to FIG. 7, the database 410 includes imaging data 418, algorithm data 412, and ML model data 416 in one aspect. Non-limiting examples of suitable imaging data 418 include resting-state functional MR imaging (rs-fMRI) data and structural MRI data as described herein that are analyzed to determine biomarker elements such as functional connectivity, cortical thickness, network efficiency, and other parameters upon which the biomarker is based. Non-limiting examples of suitable algorithm data 412 include any values of parameters defining the calculation of correlations or correlation strengths used to define functional connectivity, the matrix elements of a functional connectivity matrix, and any other relevant parameter. Non-limiting examples of ML model data 416 include any values of parameters defining the machine learning model used to predict a survival outcome based on the functional connectivity matrix of a brain tumor patient in accordance with the methods described above.

The computing device 402 also includes a number of components that perform specific tasks associated with the methods of classifying a pain patient, predicting a pain level, and/or selecting a treatment for a patient based on the multi-mode biomarker as disclosed herein. In the exemplary aspect, the computing device 402 includes a data storage device 430, an imaging component 440, a functional connectivity component 450, an ML component 470, a graph theory component 475, and a communication component 460. The data storage device 430 is configured to store data received or generated by the computing device 402, such as any of the data stored in database 410 or any outputs of processes implemented by any component of the computing device 402. The imaging component 440 is configured to operate an MRI system 810 (see FIG. 6) to obtain fMRI data and/or structural MRI data from a patient. The functional connectivity component 450 is configured to produce a functional connectivity map using the methods described herein. The ML component 470 is configured to transform the multi-mode biomarkers into predicted clinical pain parameters such as patient-reported scores using a machine learning model as described herein. The graph theory component 475 is configured to transform the functional connectivity data into graph theory parameters such as betweenness centrality, clustering coefficient, degree centrality, and local efficiency using methods as described herein.

The communication component 460 is configured to enable communications between the computing device 402 and other devices (e.g. user computing device 830 and/or MR imaging system 810 shown in FIG. 6) over a network, such as a network 850 (shown in FIG. 6), or a plurality of network connections using predefined network protocols such as TCP/IP (Transmission Control Protocol/Internet Protocol).

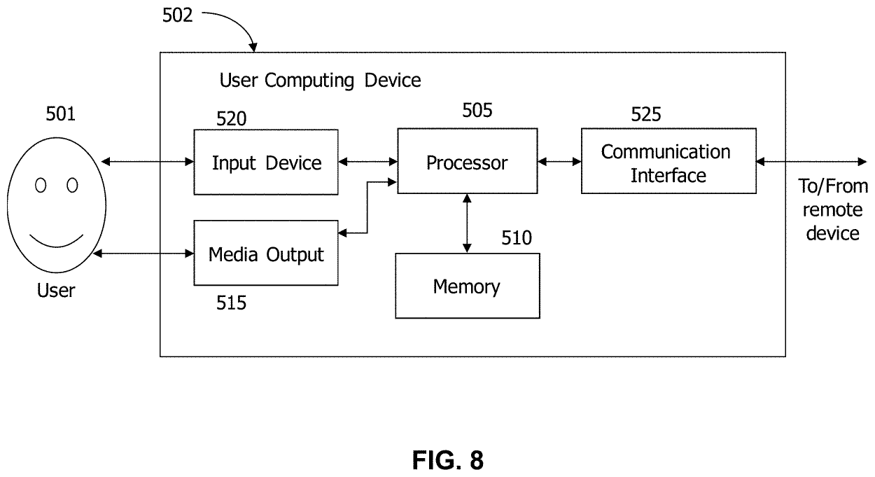

FIG. 8 depicts a configuration of a remote or user computing device 502, such as the user computing device 830 (shown in FIG. 6). The computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), an organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser, and a client application. Web browsers enable users 501 to display and interact with media and other information typically embedded on a web page or a website from a web server. A client application allows users 501 to interact with a server application associated with, for example, a vendor or business.

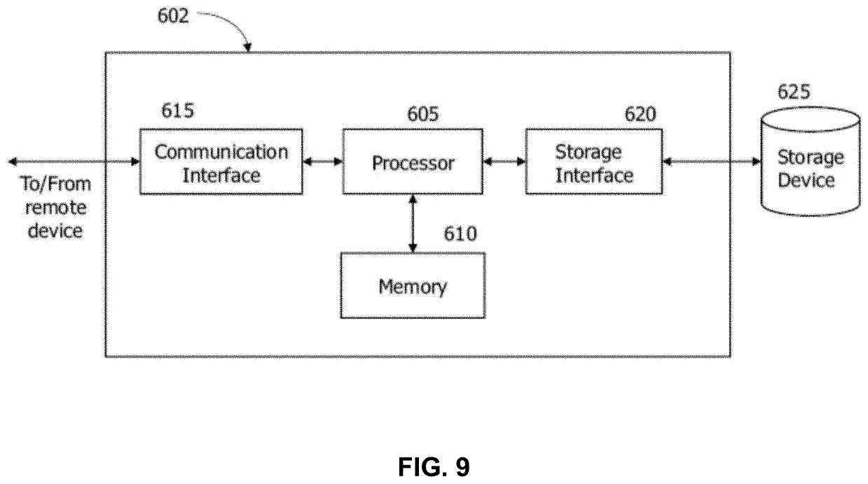

FIG. 9 illustrates an example configuration of a server system 602. Server system 602 may include, but is not limited to, database server 806 and computing device 802 (both shown in FIG. 6). In some aspects, server system 602 is similar to server system 804 (shown in FIG. 6). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 830 (shown in FIG. 6) or another server system 602. For example, communication interface 615 may receive requests from a user computing device 830 via a network 850 (shown in FIG. 6).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network-attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 8) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for the storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local, remote, or cloud-based processors, transceivers, servers, and/or sensors (such as processors, transceivers, servers, and/or sensors mounted on a vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to: images or frames of a video, object characteristics, and object categorizations. Data inputs may further include: sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a type of motion, a diagnosis based on the motion of an object, motion analysis of an object, and trained model parameters ML outputs may further include: speech recognition, image or video recognition, functional connectivity data, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above. For example, an ML module may receive training data comprising customer identification and geographic information and an associated customer category, generate a model that maps customer categories to customer identification and geographic information, and generate an ML output comprising a customer category for subsequently received data inputs including customer identification and geographic information.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In one aspect, an ML module receives unlabeled data comprising customer purchase information, customer mobile device information, and customer geolocation information, and the ML module employs an unsupervised learning method such as “clustering” to identify patterns and organize the unlabeled data into meaningful groups. The newly-organized data may be used, for example, to extract further information about a customer's spending habits.

In yet another aspect, ML methods and algorithms are directed toward reinforcement learning, which involves optimizing outputs based on feedback from a reward signal. Specifically, ML methods and algorithms directed toward reinforcement learning may receive a user-defined reward signal definition, receive data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware, or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving media, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application-specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for the storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Wash.). In yet another aspect, the system is run on a mainframe environment and a UNIX® server environment (UNIX is a registered trademark of X/Open Company Limited located in Reading, Berkshire, United Kingdom). The application is flexible and designed to run in various environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independently and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

### EXAMPLES

The following examples illustrate various aspects of the disclosure.

### Example 1: Identification of Biomarkers for Low Back Pain (LBP)

To assess the use of graph theory measures as potential biomarkers for LBP, the following experiments were conducted.

Graph theory measures were derived from resting-state functional connectivity (rsFC) measurements and evaluated as potential brain biomarkers for LBP. Resting-state functional MRI scans were collected from 24 LBP patients and 27 age-matched healthy controls (HC). We trained a support vector machine (SVM) using graph-theoretical features to classify LBP subjects from HC. The degree centrality (DC), clustering coefficient (CC), and betweenness centrality (BC) were found to be significant predictors of the patient group while using a combination of Elastic Net and optimal subset selection method (Enet-subset) method during feature selection. We achieved an average classification accuracy and AUC of 83.1% (p<0.004) and 0.937 (p<0.002), respectively. Similarly, we achieved a sensitivity and specificity of 87.0% and 79.7%. The classification results from this study suggest that graph matrices derived from rsFC can be used as biomarkers for LBP. In addition, they also prove that the proposed Enet-subset method used with this dataset has a significant impact on feature selection by removing redundant variables and reducing computational resources.

It can be difficult to identify disruptions in functional connectivity, especially in association with disorders such as chronic pain, as rsFC matrices tend to be data-rich. This problem can be addressed by using a machine learning classifier. The goal of classification learning algorithms is to build a classifier that can accurately predict an unseen test dataset by using a set of essential training features. Variable selection methods play an important role in eliminating redundant variables that directly affect prediction accuracy. Elastic Net (Enet), a hybrid algorithm of Least Absolute Shrinkage and Selection Operator (LASSO) and Ridge regression, is a widely used feature selection method. Enet is particularly useful when the number of predictors (p) is much higher than the sample size (N) (i.e. p>>N) or when there are many correlated predictor variables. However, at least a portion of the features selected by Enet from the original list of features may not always constitute the best performing subset of features. To increase the performance of a machine learning classifier, additional redundant variables can be removed. To address this, we created and tested a feature selection approach that further sorted features according to the magnitude of the absolute values of their Enet coefficients. We then selected the best subset using a nested cross-validation approach. The best subset of predictors retained in the final model was determined by the maximum cross-validated AUC, a criterion used to evaluate classifier performance. This approach to selecting an optimal subset of predictors for enhanced classifier performance is a combination of Enet with optimal subset selection extension. We refer to this new feature selection approach as Elastic Net-subset (or Enet-subset).

The aims of this study were to extract graph measures from functional connectomes and determine their ability to predict LBP by training a support vector machine to accurately classify LBP from healthy controls by using a hybrid Enet-subset feature selection technique. We collected high-resolution resting-state scans and self-reported clinical data for the Disabilities of the Arm, Shoulder and Hand (DASH) outcome measure. All MRI data were parcellated using the Human Connectome Project's (HCP) multi-modal surface-based cortical parcellation (MMP) which contains 180 symmetric cortical parcels per hemisphere. This parcellation is defined in terms of surface vertices and used across multiple modalities to define cortical areal borders, making it possible to accurately map the parcellation to individual subjects.

### I. Methods and Materials

### A. Participants

The subjects who participated in this study included 27 healthy controls (HC) and 24 LBP subjects (age-matched; p=0.21, Wilcoxon rank-sum test). All LBP subjects recruited for this study had been diagnosed with chronic LBP due to lumbar spondyloarthropathy with a history of 6 months without lower extremity symptoms. All LBP patients had not received lumbar spine surgery at the time of scanning. All HCs had no history of neurological injury or disease prior to their scan. Table 1 summarizes the participant information.

### B. Patient Inclusion and Exclusion Criteria

Low back pain (LBP) patients in the study were recruited from a patient population with a history of LBP over 6 months without lower extremity symptoms. Exclusion criteria include the following: ≤17 years old or >80 years old; pregnant; having an MRI-incompatible device; dental implants; disorders including amyotrophic lateral sclerosis, multiple sclerosis, rheumatoid arthritis, spine tumor, brain tumor, encephalopathy, traumatic brain injury, psychiatric disease, dementia, meningitis, previous incidence of SCI, or HIV-related myelopathy; having systemic instability or being deemed unable to tolerate standard MRI scanning; abnormal orientation and cranial nerve physical examination. Patients with documented learning disabilities or patients who did not undergo standard of care post-injury physical therapy were also excluded.

TABLE 1
Participants’ demographic information.

| Variable | Healthy Controls | LBP |
|---|---|---|
| Participants (n) | 27 | 24 |
| Sex (M/F) | 15/12 | 9/15 |
| Age (in years) | 46.9 ± 17.3 (25-75) | 53.5 ± 10.2 (29-67) |

### C. DASH Data Acquisition

Data for the Disabilities of the Arm, Shoulder and Hand (DASH) outcome measure was collected from each patient. The DASH outcome is a self-administered region-specific outcome instrument developed as a measure of self-rated upper-extremity disability and symptoms. The DASH score has been gaining popularity in the study of many upper extremity disorders. The two optional scales of the DASH (sport/music and work) were not included in this study. Each item in the disability/symptom scale has 5 response options. The DASH outcome measure consists mainly of a 30-item disability/symptom scale, scored 0 (no disability) to 100 (most severe disability).

D. fMRI Data Acquisition and Preprocessing

For all participants, 0.8 mm isotropic T1-weighted and T2-weighted scans were obtained using a 3T Siemens Prisma and 32-channel head coil. Resting-state fMRT images were acquired on the same day using the following parameters: Multi-band gradient-echo EPI (Multi-band accel. factor=6) with high spatial (2.4×2.4 mm×2.4 mm) and temporal (TR=800 ms) resolution (repetition time [TR]=800 ms, echo time [TE]=33 ms and flip angle=52°). The fMRI data were corrected for distortion by using a 2.4 mm isotropic spin echo field map that was matched to the fMRT acquisition.

Six resting-state fMRI scans, each approximately 5 minutes long, with AP/PA phase encoding directions (60 axial slices each) were collected. Volumetric navigator sequences were used to collect T1- and T2-weighted sequences that prospectively corrected for motion by repeating scans. While collecting the resting scans, subjects were asked to focus their attention on a visual cross-hair and remain awake.

All MRI and fMRI data were preprocessed using the Human Connectome Project's minimal preprocessing pipelines (v4.0.0) including the PreFreeSurfer, FreeSurfer, and PostFreeSurfer HCP Structural Preprocessing Pipelines for generating subcortical segmentation and cortical surfaces; functional preprocessing and denoising pipelines, which include the fMRIVolume, fMRISurface, and multi-run spatial ICA+FIX pipelines that correct for motion and distortions within fMRI data by mapping it into a standard CIFTI grayordinate space and removing spatially specific structured noise; and the MSMAll areal-feature-based cross-subject surface registration pipeline for precisely aligning the individual subjects' cortical areas to the HCP's multi-modal parcellation. The MSMAll aligned resting-state fMRI data was cleaned of global noise using temporal ICA after spatial ICA had been used to clean the data of spatially specific noise.

### E. Human Connectome Minimal Preprocessing Pipelines

The HCP PreFreeSurfer structural pipeline creates an undistorted structural volume space for each subject in which the T1- and T2-weighted images are aligned. A modified FreeSurfer pipeline segments MRI volumes into predefined structures and reconstructs cortical surfaces. The PostFreeSurfer pipeline then performs initial folding-based surface registration to an atlas using MSMSulc, computes T1w/T2w myelin maps and curvature-corrected cortical thickness maps, and produces MRI volume and surface files that can be viewed on Connectome Workbench software and prepared for further analysis.

After the structural HCP pipeline is completed, functional preprocessing pipelines begin working on the individual time series files. The fMRIVolume pipeline removes EPI distortion, spatially realigns data for motion, registers fMRI data to structural MRI, and corrects the intensity bias field. The fMRISurface pipeline brings the cortical time series from the volume onto the surface and subcortical areas into alignment with MNI space based on nonlinear volume registration to form the grayordinate space. The multi-run spatial ICA+FIX pipeline demeans, detrends, and concatenates the subject's six fMRI runs before proceeding to remove spatially specific structured noise (from subject motion physiology and the scanner) from the fMRI data. MSMSulc is used to project the fMRI data onto the 32k mesh before running MSMAll. The MSMAll surface-registration pipeline aligns cortical areas across subjects more precisely than is possible with cortical folding alone.

Temporal independent component analysis (ICA) was used to clean the MSMAll aligned resting-state fMRI data of global noise after spatial ICA had been used to clean the data of spatially specific noise (using hand classification of spatial ICA components given that FIX performance on this 2.4 mm dataset was 97%, indicating that FIX retraining was needed). Because of the relatively small size of the dataset, temporal ICA was unable to isolate a single or few global group noise components and instead found many single/few subject global components with imperfect separation of global signal and noise. Thus, instead of estimating the temporal ICA decomposition on this dataset, weighted regression of group spatial ICA components from a much larger HCP-Young Adult 1071-subject dataset with an existing temporal ICA decomposition was applied and the resulting concatenated individual subject component time courses were unmixed using the previously computed temporal ICA unmixing matrix. The noise temporal ICA individual subject component time-series from this larger dataset were then non-aggressively regressed out from the subject time-series producing similar resting-state cleanup results to those that were previously published.

### E. Graph Theory Analyses

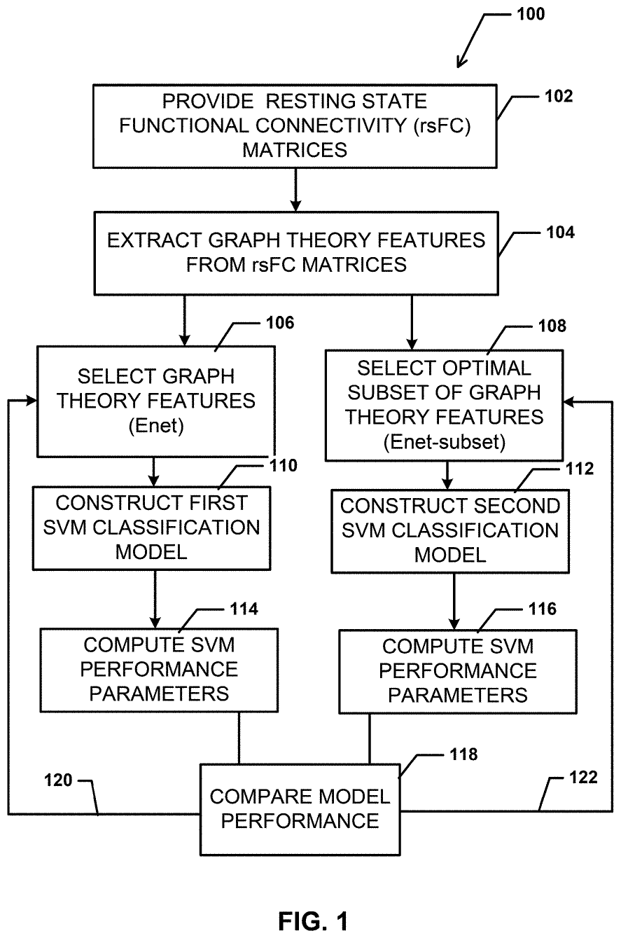

FIG. 1 is a flow chart summarizing the data processing pipeline 100 used for the development of SVM classification models based on graph theory features of resting-state functional connectivity (rsFC) matrices as described in additional detail below. Resting-state functional connectivity (rsFC) matrices are computed for each subject and provided at 102. Graph theory features are then extracted from the rsFC matrices at 104. The graph theory features included BC: Betweenness Centrality, CC: Clustering Coefficient, DC: Degree Centrality, and LE: Local Efficiency. Features of the rsFC matrices were selected using an Elastic Net feature selection method at 106 and an optimal subset selection approach was used to identify predictive features while reducing feature redundancy at 108. Two SVM models were constructed for each of the feature selection approaches at 110 and 112. Each model's performance (accuracy, AUC, sensitivity, specificity, and the total number of features used in the final model) were computed at 114 and 116 and these performance parameters were compared between both models at 118. A significance test was performed using a permutation test approach. The whole process was repeated for each feature set individually and for all combinations (for example, BC+CC+DC) at 120 and 122.

Nodes of the cortical functional network were defined as one of 360 non-overlapping parcels of the Human Connectome Project's (HCP) multi-modal surface-based cortical parcellation (MMP). The nodes from each function connectome were labeled as members of one of 12 resting-state networks (RSNs) based on the Cole-Anticevic parcellation. These RSNs were the primary visual (VIS1), secondary visual (VIS2), auditory (AUD), somatomotor (SOM), cingulo-opercular (CON), default-mode (DMN), dorsal attention (DAN), frontoparietal cognitive control (FPN), posterior multimodal (PML), ventral multimodal (VML), language (LAN), and orbito-affective (OA) networks.

To construct cortical connectivity matrices for each subject, we first took the average time-series of each of the 360 cortical areas from the preprocessed fMRI data. We then computed the Pearson's correlation coefficient between each pair of cortical areas before applying a Fisher-z transformation. Thresholding a functional connectivity matrix based on correlation strength has been shown to yield different network densities which can influence network properties that bias graph metric comparisons between patient populations. To address this potential bias, we thresholded all graphs at the same network densities and binarized the graphs prior to calculating any graph theory metrics. Binarization was used to preserve the most probable functional connections and treat those connections equivalently. As there is no universally accepted threshold for functional connectivity strength, we thresholded connections in Fisher-z transformed matrices within the top 15% for each individual, in steps of 2.5% up to 30% density, to create binary undirected graphs for each network density. These metrics were then averaged across thresholds for each node.

Using the Brain Connectivity Toolbox, we calculated the following local graph measures for each patient: clustering coefficient, local efficiency, degree centrality, and betweenness centrality. As used herein, the term “clustering coefficient” is defined as the fraction of triangles around a network and serves as a measure of how well-connected neighbors of a node are to each other. As used herein, the term “degree centrality” is defined as the number of edges for a specific node and serves as a measure of the importance of a node by assuming that the importance of a node is related to the number of nodes that it is directly connected to. As used herein, the term “betweenness centrality” is defined as a centrality measure based on shortest paths and serves as a measure of how influential a node is as information passes through it to other nodes. As used herein, the term “local efficiency” is a measure of the efficiency of information transfer within the local neighborhood of a node. The metrics described above are used to investigate network properties within the local neighborhood of a node and have been the subject of many studies of various chronic pain conditions.

### F. Machine Assisted Classification

We used a support vector machine (SVM) with a linear kernel as a classifier in this study. The pool of subject data was randomly separated into training and testing sets in a 70/30 ratio, keeping the ratio of HC to LBP patients in each group constant. The training dataset was used for the feature selection and model training phases (see FIG. 1 at 106, 108, 110, and 112) as described below. Each model's performance was tested using the testing dataset (see FIG. 1 at 114 and 116). We used the caret and glmnet packages available in RStudio for our machine learning analysis.

### Feature Selection

Each cortical parcel was modeled as a node such that 360 features were extracted for each graph theory measure. These features were then used in two different feature selection approaches that aimed to remove any redundant features to achieve both higher classification performance and better generalization to independent datasets. The first feature selection approach, Elastic Net (Enet), shrinks the coefficients of the input features to zero if they are not positively contributing. Parameter optimization was done using a grid approach on the predefined penalty parameter lambda λ=seq (0.1, 0.9, by=0.1] and α===seq ([0.0001, 0.005, by=0.001). We were constrained to a small alpha value due to the small number of features that survived (non-zero coefficients). Increasing, our alpha values would have led to the underfitting of the SVM-classifier with this data set. Following this, all the features with non-zero coefficients that formed the Enet were used as the input to the SVM classifier (see FIG. 1 at 110).

The second feature selection approach, Enet-subset, uses the coefficients estimated by Enet. The Enet-selected features were sorted in descending order based on coefficient absolute value, and a portion of the sorted features was then used to build an SVM classifier (see FIG. 1 at 112). We trained the classifier using a subset starting with the top 25 features, ranked by feature coefficient, with a step size of 25. The best subset of predictors retained in the final model was then determined by the maximum cross-validated AUC. The procedure for the Enet-subset method is summarized below:

Step #1: Sort the absolute value of Enet coefficients for selected features in descending order.

Step #2: In a loop for each subset=range [25: the total number of features, step size=25] compute AUC using an SVM linear classifier and nested 4-fold cross-validation approach.

Step #3 Determine AUC for all subsets and the select best performing subset (out of the subsets tested) for the final SVM

### Model Training and Classification

In the model training phase, features selected using Enet and the Enet-subset method were used to train two separate SVM models (see FIG. 1 at 110 and 112). As before, the features were normalized, and optimal model parameters were fed into each final SVM model. To build the best-performing SVM model, the optimized model parameters of each SVM classifier—the cost (C)—were estimated by using a grid-search algorithm. The search scale used was C=1:10. After the grid-search, the best-performing cost was used in each final model. To generalize the training process and obtain a more accurate model, we used a 4-fold (K=4) cross-validation, which was repeated 5 times. This technique divides data into equal disjointed subsets of size 4. The model was then trained on all folds except one. The remaining subset is reserved for testing purposes. This process was then repeated 3 (K−1) times, selecting each fold to be used for testing once. We repeated this process 5 times to ensure that our trained model acquired most of the patterns from the training dataset.

The performance of the SVM model was tested using a testing data set where HCs were classified as positive and LBP as negative in the true positive (TP), false positive (FP), true negative (TN), and false negative (FN) calculations. The accuracy (%) is defined as the ratio of accurately classified subjects to the total number of subjects {(TP+TN)/(TP+TN+FP+FN)}. Specificity and the sensitivity values for each model were also evaluated. Sensitivity is defined as the fraction of correctly classified positive samples from all positive samples, or the true positive rate, calculated as {TP/(TP+FN)}, and indicates the accuracy of the prediction group. Specificity is defined as the fraction of correctly classified negative samples from all negative samples, or true negative rate, calculated as {TN/(TN+FP)}, and indicates the accuracy of the prediction of the absence group. An area under the ROC (Receiver Operating Characteristics Curve) analysis was used to evaluate each model's overall performance.

### G. Statistical Tests

An unpaired two-sample Wilcoxon rank-sum test with p<0.05 was used to evaluate for statistically significant differences in group comparisons of graph measures. To correct for multiple comparisons, we used False Discovery Rate Correction (FDR) with q<0.05.

During the model training phase, the data were randomly divided into testing and training datasets which may produce slightly different models depending on the division. To address this, the two SVMs were run 100 times and the results were averaged to calculate final performance measures. The arithmetic means of the accuracy, sensitivity, specificity, and AUC of the 100 repetitions were computed for the final analysis.

Statistical significances of the classification accuracy and AUC were tested using permutation testing with 1000 permutations. For this step, the subject's class (group) was randomly assigned. The resulting accuracy produced a null-hypothesis distribution that could be used to calculate the p-value of the corresponding accuracies (i.e. the proportion of permutations that yielded a greater accuracy than the accuracy found for the classification models).

### II. Results

### A. Clinical Survey Data

We compared the LBP total DASH outcome scores to HC using a non-parametric Wilcoxon rank-sum test. There was a significant difference (p=5.21e−8; z=5.44) in the total DASH scores of LBP and HCs. Patients with chronic LBP had a higher total DASH score which was indicative of a higher disability of motor functioning in their upper extremities.

TABLE 2
DASH scores for each patient group.

| | | | Standard |
|---|---|---|---|
| Patient Group | Mean | Median | Deviation |
| Low Back Pain | 21.7 | 16.3 | 16.6 |
| Healthy Controls | 2.31 | 0 | 5.19 |

B. LBP and HCs have Similar Nodal Properties

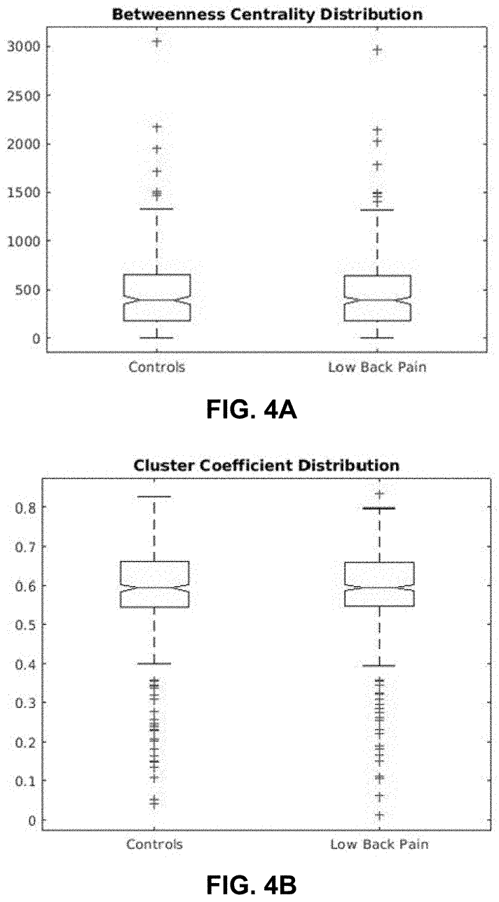

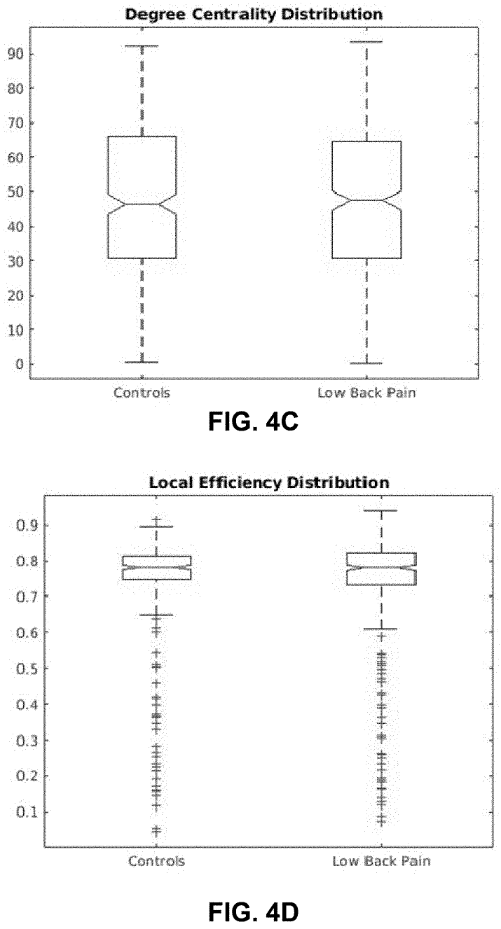

As summarized in Table 3 below, there were no significant differences in the local efficiency (LE, FIG. 4D), clustering coefficient (CC, FIG. 4B), degree centrality (DC, FIG. 4C), or betweenness centrality (BC, FIG. 4A) of nodes from the reconstructed brain networks between LBP patients and HCs after FDR correction (all p>0.05).

TABLE 3
Statistical Significance of global graph theory measures.

| | LBP | HC | Statistic (mean ± |
|---|---|---|---|
| Metric | (mean ± SD) | (mean ± SD) | standard deviation) |
| Betweenness | 456.97 ± 568.86 | 453.94 ± 576.74 | z = 0.012 ± 0.243; |
| Centrality | | | p =1 |
| Clustering | 0.586 ± 0.194 | 0.585 ± 0.194 | z = 0.0135 ± 0.28; |
| Coefficient | | | p =1 |
| Degree | 46.238 ± 30.423 | 46.798 ± 31.589 | z = −0.0134 ± 0.378; |
| Centrality | | | p =1 |
| Local | 0.742 ± 0.222 | 0.745 ± 0.209 | z = 0.167 ± 1.01; |
| Efficiency | | | p = 0.485 ± 0.295 |

The p values shown have been corrected for multiple comparisons.

### C. Machine Learning to Predict LBP

We used the BC, CC, DC, LE of all 360 parcels to train a support vector machine used to accurately classify each subject based on their respective patient group as described above and determined the matrix of best-performing features for each graph measure. We repeated this step to determine if a combination of graph measures led to a higher classification accuracy than a single measure. We achieved a maximum (mean of 100 iterations) classification accuracy of 83.1% (p<0.004), AUC of 0.94 (p<0.002), sensitivity of 87% (p<0.076), and a specificity of 79.7% (p<0.054) when using BC, CC and DC with an Enet-subset feature selection approach.

Of the four graph theory matrices used, BC, CC, and DC had very high classification accuracies with both feature selection approaches. However, LE proved to have a low classification accuracy with both feature selection approaches. We then combined BC, CC, and DC and compared their predictive power between the two feature selection methods. In all iterations, the performance of the classifier decreased slightly when using Enet features but increased when using Enet-subset features. Table 4 summarizes the overall classification results. Table 5 is a summary (mean of 100 iterations) of sensitivity and specificity using the Enet and Enet-subset feature selection methods that summarizes the sensitivity and specificity of each biomarker obtained using each feature selection method.

TABLE 4
A summary (mean of 100 iterations) of the classification accuracy
and AUC using the Enet and Enet-subset feature selection methods.

| | Using all Enet | | Using Enet-subset | |
|---|---|---|---|---|
| | selected features | | selected features | |
| | | Features | | Features |
| Bio- | ACC (%), | (mean/ | ACC (%), | (mean/ |
| marker(s) | AUC (mean) | total #) | AUC (mean) | total #) |
| BC | 81.7, 0.919 | 349/360 | 82.6, 0.920 | 326/360 |
| CC | 81.0, 0.92 | 349/360 | 82.3, 0.925 | 328/360 |
| DC | 80.9, 0.898 | 348/360 | 81.2, 0.895 | 324/360 |
| LE | 50.8, 0.598 | 348/360 | 50.4, 0.590 | 155/360 |
| BC + CC | 81.0, 0.923 | 679/720 | 82.5, 0.92 | 634/720 |
| BC + DC | 81.2, 0.907 | 680/720 | 83.2, 0.924 | 636/720 |
| CC + DC | 80.8, 0.913 | 680/720 | 81.8, 0.921 | 640/720 |
| BC + CC + DC | 80.9, 0.916 | 1006/1080 | 83.1, 0.937 | 945/1080 |

ACC: Accuracy;
AUC; Area under curve;
BC: Betweenness centrality;
CC: Clustering coefficient;
DC: Degree centrality;
LE: Local efficiency.

TABLE 5
Sensitivity and Specificity of Enet and
Enet-subset feature selection approaches.

| | Using all Enet | | Using Enet-subset | |
|---|---|---|---|---|
| | selected features | | selected features | |
| | SEN (%), | Features | SEN (%), | Features |
| Bio- | SPE (%) | (mean/ | SPE (%) | (mean/ |
| marker(s) | (mean) | total #) | (mean) | total #) |
| BC | 85.4, 78.5 | 349/360 | 85.0, 80.6 | 326/360 |
| CC | 84.0, 78.4 | 349/360 | 84.2, 80.5 | 328/360 |
| DC | 82.5, 79.5 | 348/360 | 83.0, 79.6 | 324/360 |
| LE | 28.5, 70.5 | 348/360 | 42.0, 57.8 | 155/360 |
| BC + CC | 85.6, 77.0 | 679/720 | 85.2, 80.1 | 634/720 |
| BC + DC | 84.8, 78.0 | 680/720 | 85.8, 81.0 | 636/720 |
| CC + DC | 84.1, 78.0 | 680/720 | 82.7, 81.1 | 640/720 |
| BC + CC + DC | 85.2, 77.1 | 1006/1080 | 87.0, 79.7 | 945/1080 |

SPE: Specificity;
SEN = Sensitivity;
BC: Between centrality;
CC: Clustering coefficient;
DC: Degree centrality;
LE: Local efficiency.

Overall, the performance and prediction accuracy of the proposed Enet-subset feature selection approach is higher in all instances when compared to using Enet alone. It is important to note that the total number of selected features used in the final models was always less when using an Enet-subset feature selection approach with a better model performance (except for LE). This supports our hypothesis that the Enet-subset method performs better at removing redundant features. This effect is most noticeable when the total number of features used is relatively large (for example using 360 features from BC vs using 1080 features by combining features from BC+CC+DC).

### D. Frequently Selected Features

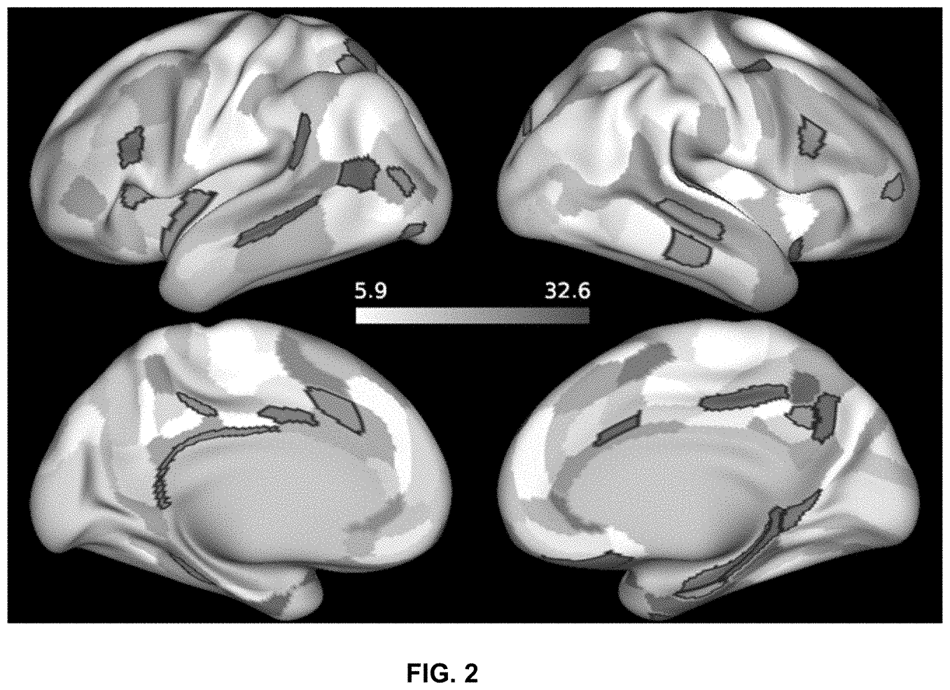

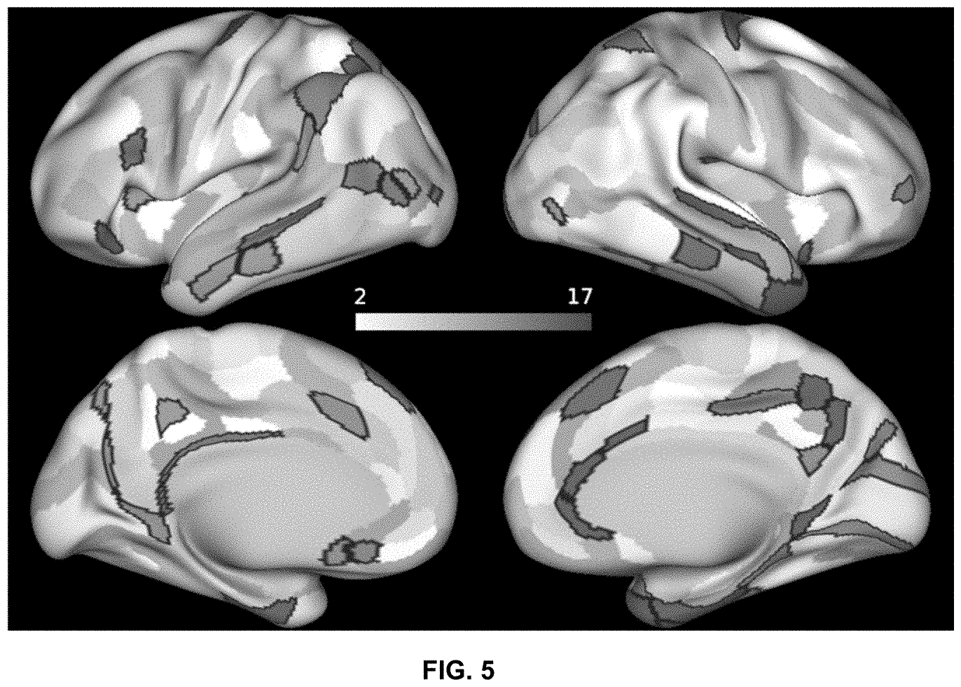

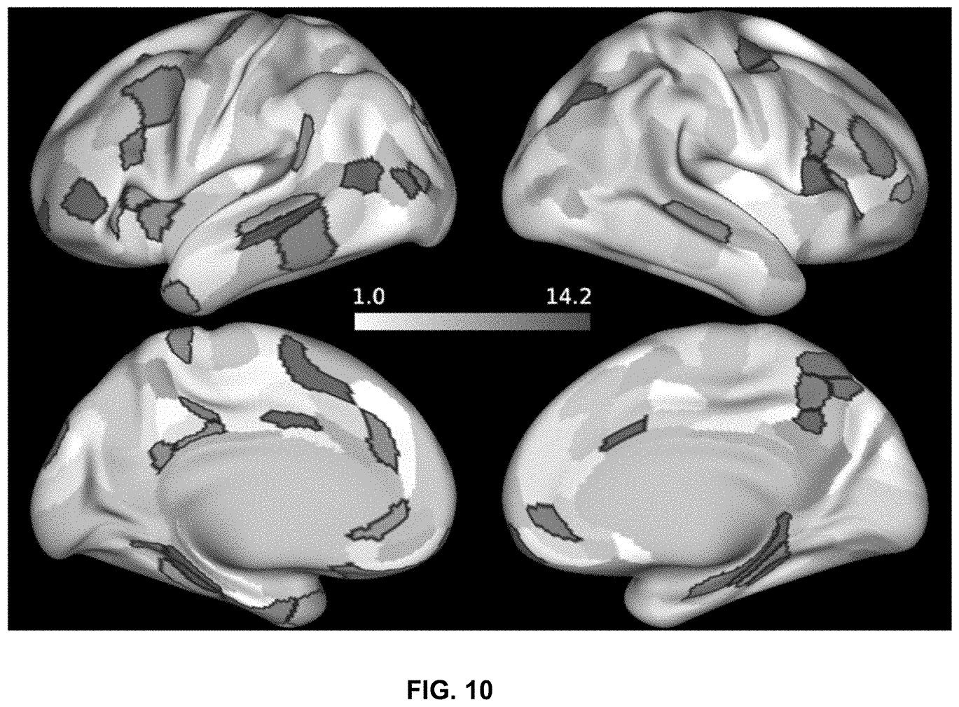

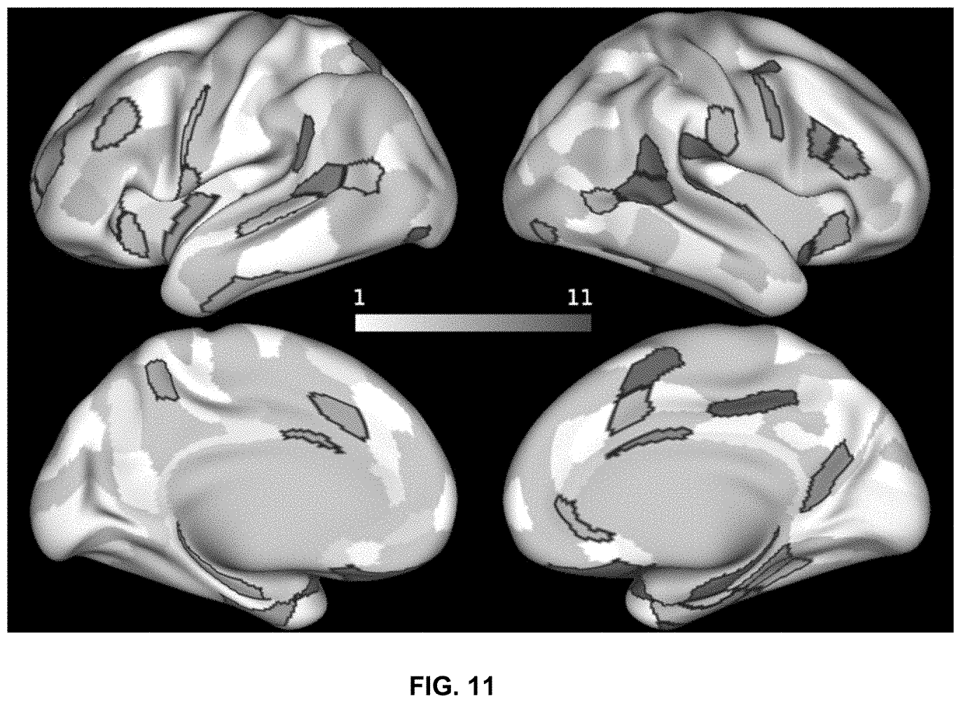

To further understand the role of individual parcels in classification performance, we saved the top 60 features (ranked by frequency) of the best performing SVM classifier (BC, CC, and DC were used as features and Enet-subset was used for feature selection during each iteration). We then sorted the parcels according to their frequency of repetition. The top 60 frequently selected cortical areas contributing to the classification and their corresponding frequency values were plotted on a brain mesh surface (FIG. 2, and see Table 6 for more details on individual parcels). In addition, we plotted the top 60 frequently selected cortical areas that contributed to the classification of each individual graph measure. FIGS. 5, 10, and 11, and Tables 6, 7, 8, and 9 below provide additional results for individual parcels.

TABLE 6
Top 60 cortical areas contributing to
classification accuracy of BC, CC, and DC combined.

| Parcel | Area | | Resting-State | |
|---|---|---|---|---|
| Number | Name | Area Description | Network | Hemisphere |
| 12 | 55b | Area 55b | Language | R |
| 14 | RSC | RetroSplenial Complex | Frontoparietal | L |
| 16 | V7 | Seventh Visual Area | Secondary | R |
| | | | Visual | |
| 22 | PIT | Posterior InferoTemporal | Secondary | L |
| | | Complex | Visual | |
| 23 | MT | Middle | Secondary | L |
| | | Temporal Area | Visual | |
| 25 | PSL | PeriSylvian | Cingulo- | L |
| | | Language Area | Opercular | |
| 27 | PCV | PreCuneus | Posterior- | R |
| | | Visual Area | Multimodal | |
| — | — | — | Posterior- | L |
| | | | Multimodal | |
| 30 | 7m | Area 7m | Default Mode | R |
| 38 | 23c | Area 23c | Cingulo- | R |
| | | | Opercular | |
| 43 | SCEF | Supplementary and | Cingulo- | R |
| | | Cingulate Eye Field | Opercular | |
| — | — | — | Cingulo- | L |
| | | | Opercular | |
| 50 | MIP | Medial IntraParietal Area | Dorsal | L |
| | | | Attention | |
| 54 | 6d | Dorsal area 6 | Somatomotor | R |
| — | — | — | Somatomotor | L |
| 57 | p24pr | Area Posterior 24 prime | Cingulo- | L |
| | | | Opercular | |
| 59 | a24pr | Anterior 24 prime | Cingulo- | R |
| | | | Opercular | |
| 60 | p32pr | Area p32 prime | Cingulo | L |
| | | | Opercular | |
| 61 | a24 | Area a24 | Default Mode | R |
| — | — | — | Default Mode | L |
| 71 | 9p | Area 9 Posterior | Default Mode | R |
| 79 | IFJa | Area IFJa | Language | R |
| 81 | IFSp | Area IFSp | Frontoparietal | L |
| 85 | a9-46v | Area anterior 9-46v | Frontoparietal | R |
| 90 | 10pp | Polar 10p | Default Mode | R |
| — | — | — | Default Mode | L |
| 93 | OFC | Orbital Frontal Complex | Default Mode | R |
| 103 | 52 | Area 52 | Auditory | R |
| — | — | — | Auditory | L |
| 106 | PoI2 | Posterior Insular Area 2 | Cingulo- | L |
| | | | Opercular | |
| 107 | TA2 | Area TA2 | Auditory | R |
| 108 | FOP4 | Frontal OPercular Area 4 | Cingulo- | L |
| | | | Opercular | |
| 110 | Pir | Pirform Cortex | Orbito- | R |
| | | | Affective | |
| — | — | — | Orbito- | L |
| | | | Affective | |
| 112 | AAIC | Anterior Agranular Insula | Orbito- | R |
| | | Complex | Affective | |
| 118 | EC | Entorhinal Cortex | Default Mode | R |
| 119 | PreS | PreSubiculum | Default Mode | R |
| 120 | H | Hippocampus | Default Mode | R |
| 121 | ProS | ProStriate Area | Primary | R |
| | | | Visual | |
| 122 | PeEc | Perirhinal Ectorhinal | Ventral- | R |
| | | Cortex | Multimodal | |
| — | — | — | Ventral- | L |
| | | | Multimodal | |
| 123 | STGa | Area STGa | Language | R |
| — | — | — | Language | L |
| 124 | PBelt | ParaBelt Complex | Auditory | R |
| 126 | PHA1 | ParaHippocampal Area 1 | Default Mode | R |
| — | — | — | Default Mode | L |
| 129 | STSdp | Area STSd posterior | Language | R |
| 130 | STSvp | Area STSv posterior | Default Mode | L |
| 136 | TE2p | Area TE2 posterior | Dorsal | R |
| | | | Attention | |
| — | — | — | Dorsal | L |
| | | | Attention | |
| 139 | TPOJ1 | Area | Language | R |
| | | TemporoParietoOccipital | | |
| | | Junction 1 | | |
| — | — | — | Language | L |
| 140 | TPOJ2 | AreaTemporoParietoOccipital | Posterior- | L |
| | | Junction 2 | Multimodal | |
| 145 | IP1 | Area IntraParietal 1 | Frontoparietal | L |
| 155 | PHA2 | ParaHippocampal Area 2 | Default Mode | L |
| 161 | 31pd | Area 31pd | Default Mode | R |
| 162 | 31a | Area 31a | Frontoparietal | L |
| 172 | TGy | Area TG Ventral | Language | R |
| 174 | LBelt | Lateral Belt Complex | Auditory | R |
| 177 | TE1m | Area TEl Middle | Default Mode | R |

TABLE 7
Top 60 cortical areas contributing to classification accuracy of BC.

| Parcel | Area | | Resting-State | |
|---|---|---|---|---|
| Number | Name | Area Description | Network | Hemisphere |
| 2 | MST | Medial Superior | Secondary | L |
| | | Temporal Area | Visual | |
| 3 | V6 | Sixth Visual Area | Secondary | R |
| | | | Visual | |
| 4 | V2 | Second Visual Area | Secondary | R |
| | | | Visual | |
| 14 | RSC | RetroSplenial Complex | Frontoparietal | L |
| 16 | V7 | Seventh Visual Area | Secondary | R |
| | | | Visual | |
| 20 | LO1 | Area Lateral Occipital 1 | Secondary | L |
| | | | Visual | |
| 23 | MT | Middle Temporal Area | Secondary | L |
| | | | Visual | |
| 24 | A1 | Primary Auditory Cortex | Auditory | L |
| 25 | PSL | PeriSylvian Language | Cingulo- | L |
| | | Area | Opercular | |
| 27 | PCV | PreCuneus Visual Area | Posterior- | R |
| | | | Multimodal | |
| 30 | 7m | Area 7m | Default Mode | R |
| 33 | v23ab | Area ventral 23 a + b | Default Mode | R |
| 38 | 23c | Area 23c | Cingulo- | R |
| | | | Opercular | |
| 47 | 7PC | Area 7PC | Somatomotor | R |
| 50 | MIP | Medial IntraParietal Area | Dorsal | L |
| | | | Attention | |
| 54 | 6d | Dorsal area 6 | Somatomotor | R |
| — | — | — | Somatomotor | L |
| 59 | a24pr | Anterior 24 prime | Cingulo- | R |
| | | | Opercular | |
| 60 | p32pr | Area p32 prime | Cingulo- | L |
| | | | Opercular | |
| 61 | a24 | Area a24 | Default Mode | R |
| 63 | 8BM | Area 8BM | Frontoparietal | R |
| 70 | 8BL | Area 8B Lateral | Default Mode | L |
| 71 | 9p | Area 9 Posterior | Default Mode | R |
| 76 | 471 | Area 471 (47 lateral) | Default Mode | L |
| 81 | IFSp | Area IFSp | Frontoparietal | L |
| 85 | a9-46v | Area anterior 9-46v | Frontoparietal | R |
| 101 | OP1 | Area OP1/SII | Somatomotor | R |
| 103 | 52 | Area 52 | Auditory | L |
| 107 | TA2 | Area TA2 | Auditory | R |
| — | — | — | Auditory | L |
| 108 | FOP4 | Frontal OPercular | Cingulo- | L |
| | | Area 4 | Opercular | |
| 110 | Pir | Pirform Cortex | Orbito- | R |
| | | | Affective | |
| 112 | AAIC | Anterior Agranular | Orbito- | R |
| | | Insula Complex | Affective | |
| 121 | ProS | ProStriate Area | Primary | R |
| | | | Visual | |
| — | — | — | Primary | L |
| | | | Visual | |
| 122 | PeEc | Perirhinal Ectorhinal | Ventral- | R |
| | | Cortex | Multimodal | |
| — | — | — | Ventral- | L |
| | | | Multimodal | |
| 123 | STGa | Area STGa | Language | R |
| — | — | — | Language | L |
| 125 | A5 | Auditory 5 Complex | Language | R |
| 126 | PHA1 | ParaHippocampal | Default Mode | R |
| | | Area 1 | | |
| 130 | STSvp | Area STSv posterior | Default Mode | L |
| 131 | TGd | Area TG dorsal | Default Mode | R |
| 132 | TE1a | Area TEl anterior | Default Mode | L |
| 135 | TF | Area TF | Ventral- | R |
| | | | Multimodal | |
| 136 | TE2p | Area TE2 posterior | Dorsal | R |
| | | | Attention | |
| — | — | — | Dorsal | L |
| | | | Attention | |
| 140 | TPOJ2 | Area | Posterior- | L |
| | | TemporoParietoOccipital | Multimodal | |
| | | Junction 2 | | |
| 142 | DVT | Dorsal Transitional | Primary | L |
| | | Visual Area | Visual | |
| 145 | IP1 | Area IntraParietal 1 | Frontoparietal | L |
| 149 | PFm | Area PFm Complex | Frontoparietal | L |
| 156 | V4t | Area V4t | Secondary | R |
| | | | Visual | |
| 161 | 31pd | Area 31pd | Default Mode | L |
| 164 | 25 | Area 25 | Default Mode | L |
| 165 | s32 | Area s32 | Default Mode | L |
| 172 | TGv | Area TG Ventral | Language | R |
| 176 | STSva | Area STSv anterior | Default Mode | R |
| 177 | TE1m | Area TE1 Middle | Default Mode | R |
| — | — | — | Frontoparietal | L |
| 180 | p24 | Area posterior 24 | Cingulo- | R |
| | | | Opercular | |

TABLE 8
TOP 60 CORTICAL AREAS CONTRIBUTING
TO CLASSIFICATION ACCURACY OF CC.

| Parcel | Area | | Resting-State | |
|---|---|---|---|---|
| Number | Name | Area Description | Network | Hemisphere |
| 10 | FEF | Frontal Eye Fields | Cingulo- | R |
| | | | Opercular | |
| 12 | 55b | Area 55b | Language | R |
| 13 | V3A | Area V3A | Secondary | L |
| | | | Visual | |
| 16 | V7 | Seventh | Secondary | L |
| | | Visual Area | Visual | |
| 23 | MT | Middle | Secondary | L |
| | | Temporal Area | Visual | |
| 25 | PSL | PeriSylvian | Cingulo- | L |
| | | Language Area | Opercular | |
| 27 | PCV | PreCuneus | Posterior- | R |
| | | Visual Area | Multimodal | |
| 29 | 7Pm | Medial Area 7P | Frontoparietal | R |
| 33 | v23ab | Area ventral 23 a + b | Default Mode | L |
| 34 | d23ab | Area dorsal 23 a + b | Default Mode | L |
| 39 | 5L | Area 5L | Somatomotor | L |
| 43 | SCEF | Supplementary and | Cingulo- | L |
| | | Cingulate EyeField | Opercular | |
| 45 | 7Am | MedialArea 7A | Cingulo- | R |
| | | | Opercular | |
| 49 | VIP | Ventral IntraParietal | Secondary | L |
| | | Complex | Visual | |
| 54 | 6d | Dorsal area 6 | Somatomotor | L |
| 57 | p24pr | Area Posterior | Cingulo- | L |
| | | 24 prime | Opercular | |
| 59 | a24pr | Anterior | Cingulo- | R |
| | | 24 prime | Opercular | |
| 61 | a24 | Area a24 | Default Mode | L |
| 64 | p32 | Area p32 | Default Mode | R |
| 73 | 8C | Area 8C | Frontoparietal | L |
| 74 | 44 | Area 44 | Language | R |
| 75 | 45 | Area 45 | Language | R |
| 79 | IFJa | Area IFJa | Language | R |
| 81 | IFSp | Area IFSp | Frontoparietal | L |
| 83 | p9-46v | Area posterior 9-46v | Frontoparietal | R |
| 85 | a9-46v | Area anterior 9-46v | Frontoparietal | R |
| 89 | a10p | Area anterior 10p | Frontoparietal | R |
| — | — | — | Frontoparietal | L |
| 90 | 10pp | Polar 10p | Default Mode | R |
| — | — | — | Default Mode | L |
| 93 | OFC | Orbital Frontal | Frontoparietal | L |
| | | Complex | | |
| 97 | i6-8 | Inferior 6-8 | Frontoparietal | L |
| | | Transitional | | |
| | | Area | | |
| 103 | 52 | Area 52 | Auditory | R |
| — | — | — | Auditory | L |
| 108 | FOP4 | Frontal OPercular | Cingulo- | L |
| | | Area 4 | Opercular | |
| 109 | MI | Middle Insular | Cingulo- | L |
| | | Area | Opercular | |
| 114 | FOP3 | Frontal OPercular | Cingulo- | L |
| | | Area 3 | Opercular | |
| 119 | PreS | PreSubiculum | Default Mode | R |
| 120 | H | Hippocampus | Default Mode | R |
| 122 | PeEc | Perirhinal Ectorhinal | Ventral- | L |
| | | Cortex | Multimodal | |
| 126 | PHA1 | ParaHippocampal | Default Mode | R |
| | | Area 1 | | |
| — | — | — | Default Mode | L |
| 127 | PHA3 | ParaHippocampal | Dorsal | L |
| | | Area 3 | Attention | |
| 129 | STSdp | Area STSd posterior | Language | R |
| — | — | — | Language | L |
| 130 | STSvp | Area STSv posterior | Default Mode | L |
| 131 | TGd | Area TG dorsal | Default Mode | L |
| 133 | TE1p | Area TE1 posterior | Frontoparietal | L |
| 140 | TPOJ2 | Area | Posterior- | L |
| | | TemporoParietoOccipital | Multimodal | |
| | | Junction 2 | | |
| 145 | IP1 | Area IntraParietal 1 | Frontoparietal | R |
| 153 | VMV1 | VentroMedial Visual | Secondary | L |
| | | Area 1 | Visual | |
| 155 | PHA2 | ParaHippocampal | Default Mode | R |
| | | Area 2 | | |
| — | — | — | Default Mode | L |
| 159 | LO3 | Area Lateral | Secondary | L |
| | | Occipital 3 | Visual | |
| 161 | 31pd | Area 31pd | Default Mode | R |
| 162 | 31a | Area 31a | Frontoparietal | L |
| 169 | FOP5 | Area Frontal | Cingulo- | L |
| | | Opercular 5 | Opercular | |
| 171 | p47r | Area posterior 47r | Frontoparietal | L |
| 174 | LBelt | Lateral Belt Complex | Auditory | R |
| 179 | a32pr | Area anterior | Cingulo- | L |
| | | 32 prime | Opercular | |

TABLE 9
Top 60 cortical areas contributing to classification accuracy of DC.

| Parcel | Area | | Resting-State | |
|---|---|---|---|---|
| Number | Name | Area Description | Network | Hemisphere |
| 12 | 55b | Area 55b | Language | R |
| 22 | PIT | Posterior | Secondary | R |
| | | InferoTemporal | Visual | |
| | | Complex | | |
| — | — | — | Secondary | L |
| | | | Visual | |
| 25 | PSL | PeriSylvian | Cingulo- | L |
| | | Language Area | Opercular | |
| 27 | PCV | PreCuneus | Posterior- | L |
| | | Visual Area | Multimodal | |
| 28 | STV | Superior Temporal | Language | R |
| | | Visual Area | | |
| 31 | POS1 | Parieto-Occipital | Default Mode | R |
| | | Sulcus Area 1 | | |
| 38 | 23c | Area 23c | Cingulo- | R |
| | | | Opercular | |
| 43 | SCEF | Supplementary and | Cingulo- | R |
| | | Cingulate Eye Field | Opercular | |
| 50 | MIP | Medial IntraParietal | Dorsal | L |
| | | Area | Attention | |
| 56 | 6v | Ventral Area 6 | Somatomotor | R |
| — | — | — | Somatomotor | L |
| 58 | 33pr | Area 33 prime | Cingulo- | R |
| | | | Opercular | |
| — | — | — | Frontoparietal | L |
| 60 | p32pr | Area p32 prime | Cingulo- | R |
| | | | Opercular | |
| — | | — | Cingulo- | L |
| | | | Opercular | |
| 61 | a24 | Area a24 | Default Mode | R |
| 79 | IFJa | Area IFJa | Language | R |
| 81 | IFSp | Area IFSp | Language | R |
| 83 | p9-46v | Area posterior 9-46v | Frontoparietal | L |
| 86 | 9-46d | Area 9-46d | Cingulo- | L |
| | | | Opercular | |
| 92 | 131 | Area 131 | Frontoparietal | L |
| 93 | OFC | Orbital Frontal Complex | Default Mode | R |
| — | — | — | Frontoparietal | L |
| 99 | 43 | Area 43 | Cingulo- | L |
| | | | Opercular | |
| 103 | 52 | Area 52 | Auditory | R |
| — | — | — | Auditory | L |
| 105 | PFcm | Area PFcm | Cingulo- | R |
| | | | Opercular | |
| 106 | PoI2 | Posterior Insular | Cingulo- | L |
| | | Area 2 | Opercular | |
| 107 | TA2 | Area TA2 | Auditory | R |
| 110 | Pir | Pirform Cortex | Orbito- | R |
| | | | Affective | |
| — | — | — | Orbito- | L |
| | | | Affective | |
| 111 | AVI | Anterior Ventral | Frontoparietal | R |
| | | Insular Area | | |
| — | — | — | Frontoparietal | L |
| 112 | AAIC | Anterior Agranular | Orbito- | R |
| | | Insula Complex | Affective | |
| 118 | EC | Entorhinal Cortex | Default Mode | R |
| 120 | H | Hippocampus | Default Mode | R |
| — | — | — | Default Mode | L |
| 122 | PeEc | Perirhinal Ectorhinal | Ventral- | R |
| | | Cortex | Multimodal | |
| — | — | — | Ventral- | L |
| | | | Multimodal | |
| 124 | PBelt | ParaBelt Complex | Auditory | R |
| 126 | PHA1 | ParaHippocampal | Default Mode | R |
| | | Area 1 | | |
| 127 | PHA3 | ParaHippocampal | Dorsal | R |
| | | Area 3 | Attention | |
| 129 | STSdp | Area STSd posterior | Language | L |
| 134 | TE2a | Area TE2 anterior | Default Mode | L |
| 135 | TF | Area TF | Ventral- | R |
| | | | Multimodal | |
| 136 | TE2p | Area TE2 posterior | Dorsal | R |
| | | | Attention | |
| — | — | — | Dorsal | L |
| | | | Attention | |
| 139 | TPOJ1 | Area | Language | R |
| | | TemporoParietoOccipital | | |
| | | Junction 1 | | |
| — | — | — | Language | L |
| 140 | TPOJ2 | Area | Posterior- | R |
| | | TemporoParietoOccipital | Multimodal | |
| | | Junction 2 | | |
| — | — | — | Posterior- | L |
| | | | Multimodal | |
| 147 | PFop | Area PF opercular | Cingulo- | R |
| | | | Opercular | |
| 166 | pOFC | posterior | Orbito- | R |
| | | OFC Complex | Affective | |
| 167 | PoIl | Area Posterior | Cingulo- | R |
| | | Insular 1 | Opercular | |
| 169 | FOP5 | Area Frontal | Cingulo- | L |
| | | Opercular 5 | Opercular | |
| 170 | p10p | Area posterior 10p | Frontoparietal | L |
| 172 | TGy | Area TG Ventral | Language | R |
| 173 | MBelt | Medial Belt Complex | Auditory | L |
| 178 | PI | Para-Insular Area | Cingulo- | R |
| | | | Opercular | |

We also conducted a Pearson's correlation test to determine any correlations between the graph measures of the top 60 frequently selected cortical parcels (see FIG. 2 and Table 6) and the patient's corresponding total DASH scores. However, we did not find any significant correlations between these graph measures and the calculated total DASH scores.

### Discussion

The literature has shown that a high level of functional interaction between cortical areas is necessary to cope with the demand of cognitive activities. We used noninvasive imaging in this study to model these functional interactions and measure network properties. The results validated our hypothesis that the use of certain graph measures as a biomarker may lead to the integration of more effective information of pain states like LBP. The results of these experiments further support the Enet-subset method as a more effective feature selection algorithm in removing redundant variables and improving the classifier's performance. Upon looking at the graph analysis as a whole, we found a lack of significant differences in individual cortical areas between HCs and LBP patients. However, the success we have seen with the machine learning models supports the notion, that groups of cortical regions are more predictive of the patient group than individual cortical regions.

### A. Predictive Cortical Areas are Involved in Spatio-Temporal Processing and its Associated Visual and Motor Coordination

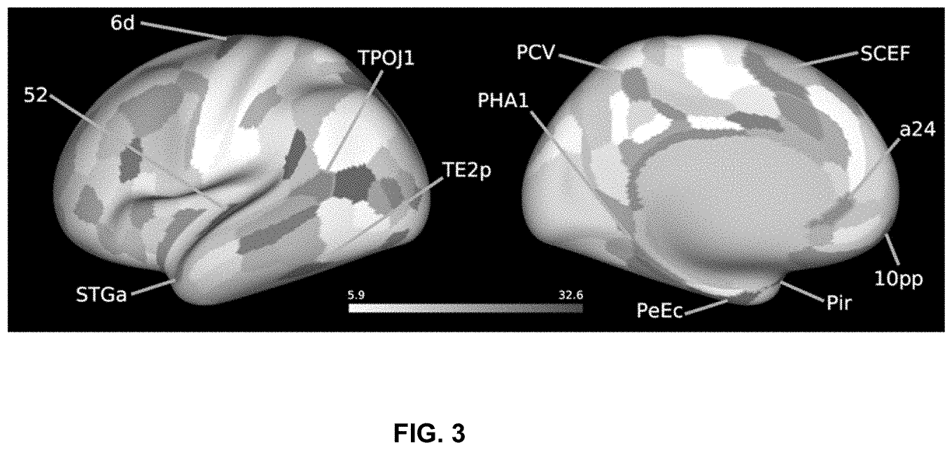

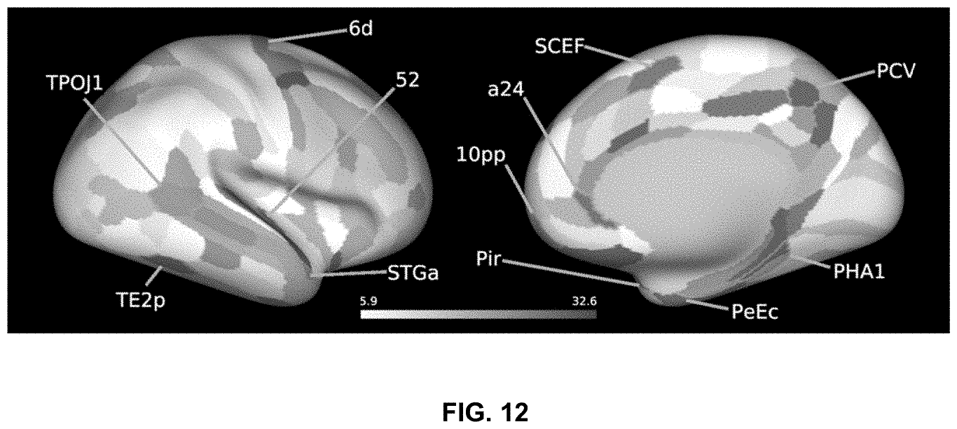

The Enet-subset model selected several bilateral cortical regions (FIG. 3, Table 10, and FIG. 12) as frequent predictive features that are necessary for spatial navigation. This is a complex process that involves the processing of multiple incoming sensory stimuli based on surrounding spatial landmarks to determine the optimal route to a specific goal.

TABLE 10
A summary of the bilateral regions from the top 60 cortical areas,
selected for by frequency that contributed to the classification accuracy
of the Enet-subset model when trained using the betweenness centrality,
degree centrality, and clustering coefficient graph measures.

| Area Name | Area Description |
|---|---|
| PCV | Precuneus Visual Area |
| SCEF | Supplementary and Cingulate Eye Field |
| 6d | Dorsal area 6 |
| a24 | Area a24 |
| 10pp | Polar 10p (Orbitofrontal cortex) |
| 52 | Area 52 (Parainsular area) |
| Pir | Pirform Cortex (Olfactory) |
| PeEc | Perirhinal Ectorhinal Cortex |
| STGa | Area STGa (Auditory) |
| PHA1 | ParaHippocampal Area 1 |
| TE2p | Area TE2 posterior |
| TPOJ1 | Area TemporoParietoOccipital Junction 1 |

The temporal-parietal-occipital junction (TPOJ) has been implicated in numerous functions such as attentional reorienting, event timing, detection of transitioning between sensory modalities, visual awareness, and the integration of these different sensory inputs. The precuneus visual area plays an important role in spatial navigation and spatial processing. Previous studies have shown that damage to this part of the parietal cortex leads to deficits in neglect, including representational space, simultagnosia, and oculomotor apraxia, all of which are related to visuospatial processing. It is possible that, although the precuneus is not directly involved in the cortical representation of pain, it predicts how likely we are to interpret external events as painful.

The Supplementary and Cingulate Eye Field (SCEF) is a part of the supplementary motor complex that is associated with the regulation of eye movement. The SCEF has anatomical connections to the frontal eye field, superior colliculus, and lateral intraparietal cortex which puts it in a unique position to regulate goal-directed behavior. The dorsal area is a part of the dorsal premotor cortex (DPC) that is also implicated in goal-directed actions that involve the positioning of the target object, hand, and eyes. Inhibiting activity of the DPC using transcranial magnetic stimulation in human patients increases reaction times which supports its role in motor planning. These findings are bolstered by the significant decline in upper extremity motor functioning shown by the differences in total DASH scores between both patient groups.

The ParaHippocampal Area (PHA) is a subregion of the ParaHippocampal cortex (PHC) and is reported to be involved in visuospatial processing including place perception and spatial representation. Individuals with lesions to the PHC show impaired visuospatial processing and difficulties with spatial orientation, navigation, and landmark identification. Area a24, a part of the anterior cingulate cortex (ACC), has been reported to show vestibular activations. In addition, there is growing evidence that spatial memories may become supported by certain extrahippocampal structures over time. The ACC is believed to be one of these structures that stores past spatial memories.

The perirhinal cortex region adds semantic knowledge to aid in item identification. In addition, the perirhinal cortex integrates item information with spatio-temporal information and transmits this data to the hippocampus via the entorhinal cortex. The temporal area 2 posterior (TE2p) cortical area is a newly identified cortical area that lies on the inferior temporal gyrus and may play a role in visual pathways, specifically in object recognition.

These bilaterally affected regions are essential in the coordination of motor control and other sensory processes necessary to facilitate spatial navigation. Studies have shown that physical self-awareness and perception of one's relative position are impaired in patients with severe chronic LBP. This evidence compounded by the downstream hand and shoulder motor deficits, as shown by differences in patient DASH scores, further supports the predictive features selected by our model.

### B. Feature Selection Using Enet-Subset is More Efficient

The Least Absolute Shrinkage and Selection Operator (LASSO) is a popular method to identify a small number of informative features. This is because of its ability to zero the coefficients of non-informative features and assign positive or negative coefficients to more informative features. However, the maximum number of features that LASSO is capable of selecting is less than the total sample size. As a result, LASSO is an ineffective option when many features are required to train the classifier LASSO. We encountered this problem with our dataset when applying LASSO. In many of its iterations (out of 100), LASSO selected very few features even after optimizing the penalty parameter (4 This led to the underfitting of our models, resulting in poor model performance.

We then applied Enet, a feature selection method based on a relatively sparse model, to select for significant variables within each graph measure. However, it was apparent that Enet still selected redundant variables. This was observed when features selected from the Enet-subset feature selection method performed better with fewer features than those selected by Enet. These redundant variables were removed to improve the accuracy of the classifier. Redundant variables lead to overfitting, low prediction accuracy, and an increase in calculation load which was computationally expensive. The proposed Enet-subset method further selected for significant variables based on each feature's coefficient from Enet. An important finding from this study was the usefulness of the Enet-subset method in reducing non-informative features and therefore increasing a model's performance (see Table 4). Additionally, this Enet-subset method was effective in reducing model complexity and calculation load with complex neuroimaging data.

### Conclusion

The results of these experiments revealed changes in graph theory metrics of resting-state fMRI in low back pain (LPB) patients relative to healthy controls, demonstrating the potential utility of graph theory features derived from resting-state fMRI as biomarkers of low back pain. A combination of an Elastic Net and Elastic Net subset selection method works better in feature selection in tandem than either selection method independently. Support vector machines were able to separate low back pain patients from healthy controls with a very high level of classification performance.

In conclusion, the highly predictive graph theory network approach used to train the classifiers supports the notion of brain function alteration in LBP. Importantly, our results also demonstrate how machine-assisted classification algorithms can accurately categorize patient-specific data into their respective cohort using graph metric matrices. This supports our hypothesis that these graph measures can be used as a biomarker of LBP. Our results also show that an Enet-subset feature selection method is more effective than a standard Enet selection method in improving a model's performance.

### Example 2: Identification of Biomarkers for Low Back Pain (LBP)

In this study, we report on morphological changes in cerebral cortical thickness (CT) and resting-state functional connectivity (rsFC) measures as potential brain biomarkers for LBP. Structural MRI scans, resting-state functional MRI scans, and self-reported clinical scores were collected from 24 LBP patients and 27 age-matched healthy controls (HC). The results suggested widespread differences in CT in LBP patients relative to HC. These differences in CT are correlated with self-reported clinical summary scores, the Physical Component Summary and Mental Component Summary scores. The primary visual, secondary visual, and default mode networks showed significant age-corrected increases in connectivity with multiple networks in LBP patients. Cortical regions classified as hubs based on their eigenvector centrality (EC) showed differences in their topology within the motor and visual processing regions. Finally, a support vector machine trained using CT to classify LBP subjects from HC achieved an average classification accuracy of 74.51%, AUC=0.787 (95% CI: 0.66-0.91). The findings from this study suggest widespread changes in CT and rsFC in patients with LBP while a machine learning algorithm trained using CT can predict patient groups. Taken together, these findings suggest that CT and rsFC may act as potential biomarkers for LBP to guide therapy.

When taken together, the literature demonstrates that LBP patients show differences on a structural and functional level within the brain. We hypothesized that patients with LBP will show disruptions in functional connectivity between brain regions involved in the processing and perception of pain. We further hypothesized that LBP patients would show aberrations in the CT within regions previously implicated in the processing of pain and that these changes would predict subject-reported clinical pain scores. Additionally, we set out to examine if variations in CT could be used as an imaging biomarker to train machine learning algorithms to classify LBP from healthy controls. Thus, the aims of this study were to characterize the cortical areas that showed age-corrected differences in cortical thickness between patient groups, determine associations between CT with self-reported clinical summary scores, characterize differences in functional connectivity on a cortical area and network level, examine global network properties and hub topology, and train a support vector machine to accurately predict LBP from healthy controls and support a clinical translation of this technique.

We collected high-resolution structural and resting-state scans and self-reported clinical data for the 36-Item Short Form healthy survey (SF-36). We used the Human Connectome Project's (HCP) multi-modal surface-based cortical parcellation (MMP) which contains 180 symmetric cortical parcels per hemisphere. This parcellation is defined in terms of surface vertices and used across multiple modalities to define cortical areal borders, making it possible to accurately map the parcellation to individual subjects.

### Methods

### A. Participants

Participants were recruited from a population of patients during hospital visits. Prior to enrollment in the study, a trained physician screened prospective participants. LBP patients with a history of LBP over 6 months without lower extremity symptoms were recruited for this study. LBP subjects had a diagnosis of chronic low back pain due to lumbar spondyloarthropathy without a history of lumbar spine surgery. All eligible healthy controls (HC) in the study had no history of neurological injury or disease at the time of scanning. A sample of 27 HC and 24 LBP subjects (age-matched; p=0.21, Wilcoxon rank-sum test) were recruited for the study.

### B. Clinical Surveys and Factor Analysis

Data for the Short-Form 36-item (SF-36) health survey questionnaire was collected from each participant. The SF-36 is summarized into 8 sub-categories 1) physical functioning (PF), 2) role limitations due to physical health problems (RLP), 3) bodily pain (P), 4) general health (GH), 5) energy fatigue (EF), 6) social functioning (SF), 7) role limitations due to emotional problems (RLE) and 8) emotional well-being (E) (Ware, 1993). A higher score for any of these categories represents a better health condition for these 8 subcategories.

These eight scales can be aggregated into physical and mental component summary scores. Scores for the eight SF-36 subscales were calculated following the standard guideline. A factor analysis approach was then applied to these scores to get the Physical Component Summary (PCS) factor score, and the Mental Component Summary score (MCS).

C. MRI and fMRI Data Acquisition and Preprocessing

All MRI data were collected in a 3T Siemens Prisma and 32-channel head coil; 0.8 mm isotropic T1-weighted and T2-weighted scans were obtained. The functional runs were collected using multi-band gradient-echo EPI (Multiband accel. factor=6). The entire brain was scanned with high spatial (2.4×2.4 mm×2.4 mm) and temporal (TR=800 ms) resolution (repetition time [TR]=800 ms, echo time [TE]=33 ms, and flip angle=52°). A 2.4 mm isotropic spin echo field map that is matched to the fMRI acquisition was obtained to correct the fMRI data for distortion. Six resting-state fMRI scans, each approximately 5 minutes long, with AP/PA phase encoding directions (60 axial slices each) were collected. T1- and T2-weighted sequences were collected using volumetric navigator sequences which prospectively corrected for motion by repeating scans. While collecting the resting scans, subjects were asked to focus their attention on a visual cross-hair and remain awake.

Preprocessing of multi-modal MRI data was done using the Human Connectome Project's minimal preprocessing pipeline (v4.0.0) including the PreFreeSurfer, FreeSurfer, and PostFreeSurfer HCP Structural Preprocessing Pipelines for generating subcortical segmentation and cortical surfaces; functional preprocessing and denoising pipelines, which include the fMRIVolume, fMRISurface, and multi-run spatial ICA+FIX pipelines that correct for motion and distortions within fMRI data by mapping it into a standard CIFTI grayordinate space and removing spatially specific structured noise; and the MSMAll areal-feature-based cross-subject surface registration pipeline for precisely aligning the individual subjects' cortical areas to the HCP's multi-modal parcellation. Temporal independent components analysis (ICA) was used to clean the MSMAll aligned resting-state fMRI data of global noise after spatial ICA had been used to clean the data of spatially specific noise.

### D. Acquisition and Analysis of Cortical Thickness (Ct) Data

To sample data at the areal level, we used the HCP's MMP. This parcellation contains 180 symmetric cortical areas per hemisphere totaling 360 parcels. For each subject, the average cortical gray matter thickness value was extracted from each of the 360 parcels that had been functionally aligned to the individual data with MSMAll. Multiple regression was used to determine if each cortical area's thickness differed significantly (p<0.05) between patients with LBP and healthy controls while controlling for age.

### E. Resting-State Functional Connectivity (Rsfc) Analysis

A functional connectome for each subject was generated by taking the average time-series in each of 360 cortical areas and taking the Fisher-z transformed Pearson's correlation between each pair of cortical areas. The function connectome was reordered so that cortical areas were grouped within one of 12 RSNs. These RSNs were the primary visual (VIS1), secondary visual (VIS2), auditory (AUD), somatomotor (SOM), cingulo-opercular (CON), default-mode (DMN), dorsal attention (DAN), frontoparietal cognitive control (FPN), posterior multimodal (PML), ventral multimodal (VML), language (LAN), and orbito-affective (OA) networks.

Differences in parcel-to-parcel connectivity were tested using a Wilcoxon rank-sum test and the corresponding z values were determined. To assess differences in connectivity between networks, the parcels of the Fisher-z transformed Pearson's correlation matrix were reorganized based on membership in a specific network and the corresponding average connectivity was computed for each network. The differences in network connectivity were then tested using a Wilcoxon rank-sum test.

### F. Graph Theoretical Analyses

Each parcel of the HCP's MMP was modeled as a node, resulting in a total of 360 non-overlapping nodes. Thresholding a connectivity matrix based on correlation strength can yield different network densities which can in turn influence network properties that bias graph metric comparisons between patient populations. Therefore, we decided to threshold all graphs at the same network densities by taking a percentage of all the positive connections and binarizing the graphs prior to calculating any graph theory metrics. Binarization is used in functional graphs to preserve only the most probable functional connections and treat these connections equivalently. As there is no accepted cutoff for functional connectivity strength to determine whether a functional connection is nontrivial, we thresholded connections in Fisher-z transformed matrices within the top 15% for each individual, in steps of 2.5% up to 30% density, to create binary undirected graphs for each network density.

Using the Brain Connectivity Toolbox (Rubinov and Sporns, 2010), we calculated the global graph metrics: global efficiency, clustering coefficient, and characteristic path length for each patient which provide an estimate of how easily information can be integrated across the network. The characteristic path length (the average smallest number of edges between all pairs of nodes in the graph that never visit a single node more than once) measures how easily information can be transferred across the network. The global efficiency (the average inverse shortest path length in the network) is a test of the ability of parallel information processing over brain networks. The clustering coefficient (the fraction of triangles around a network) is a measure of how well connected the neighbors of a node are to each other. We averaged these metrics across thresholds for each node as previously published.

We determined the network efficiency, at the global level, of each RSN for each patient by calculating its global efficiency. This provides an estimate of parallel information transformation and global functioning within a specific RSN. We extracted the thresholded and binarized connectome for each intra-network interaction at each network density and calculated the global efficiency of each RSN rsFC matrix for each patient using the Brain Connectivity Toolbox. Differences in the global efficiency of each RSN were tested using a Wilcoxon rank-sum test and the corresponding z values were determined.

### G. Identification of Hubs

Hubs can be identified using different graph theory measures such as degree (number of connections a node has) or centrality (relative importance of a node with respect to its surrounding nodes in propagating the information to other nodes in the network). Eigenvector centrality is a centrality measure of how well connected one node is to other nodes that are well connected. We chose eigenvector centrality to classify hubs due to its more self-referential nature. We calculated the eigenvector centrality for each parcel in each patient using the Brain Connectivity Toolbox. These values were then averaged across patients for each parcel to form a group average for LBP patients and HC. Hub status was assigned to nodes whose eigenvector centrality was one standard deviation above the group mean. We identified parcels that were found to be hubs in 1) both LBP patients and HC, 2) only HC and not in LBP patients, and 3) only LBP patients and not in HC.

### H. Machine Assisted Classification

A support vector machine (SVM) classifier, with a linear kernel, was used due to its established predictive power with relatively small sample sizes. We used the caret package available within RStudio (rstudio.com) to implement our machine learning classifier. We used leave-one-out (LOO) cross-validation to test the performance of our SVM due to the limited number of patients in the present study. The steps involved in the SVM classification analysis are briefly discussed below. It is important to note that the feature selection, parameter optimization, and final model training, in each LOO iteration, was performed on the training dataset which included all subject data except for one (the left-out subject or the test subject).

### Feature Reduction

We used 360 features (one cortical thickness value for each of the 360 parcels) with a relatively small sample size (subject number=51). We used a dimensionality reduction approach as the dimensions (number of features) of the data were much larger than the sample size. This method of feature selection (or reduction) is essential to reduce the high-dimensional data to a lower-dimensional subset to avoid overfitting, a common problem in neuroimaging. We aimed to keep relevant features and remove relatively insignificant feature variables to achieve a higher classification performance when testing data and a better generalization to independent datasets. We used recursive feature elimination (RFE) for this study. RFE is a popular feature selection approach that is effective in data dimension reduction, increases the efficiency of MRI datasets, and is applied in many neuroimaging studies. RFE aids in the elimination of redundant features without incurring a substantial loss of information and retains a set of the most informative features to be used in SVM model training. Within the RFE framework, we used 4-fold cross-validation with ten repetitions to get most of the data patterns from the training set and to obtain a best-predicting feature subset.

### Model Training and Classification of Test Subject(s)

In the model-training phase, RFE-selected features were used to train the SVM model. As with many other supervised machine learning approaches, the SVM algorithm performs poorly on experimental data when the default parameter values are used. Accordingly, the training set was utilized to determine the optimal parameters of the SVM classifier and to build the best-performing SVM model. The model parameter (the cost in the case of linear SVM) is optimized to maximally discriminate one group from another (HC from LBP group) by using the grid-search algorithm. In the present study, the search scale was c=1:10. After the grid-search, the best-performing cost was used in the final model. The performance of the SVM model was trialed using a testing data set (left-out subject's data) in each LOO iteration.

### Evaluation of Overall Performance (Accuracy, Sensitivity, Specificity, and AUC)

The output of a binary classifier is viewed as a confusion matrix. The accuracy percentage (%) is defined as the ratio of the number of accurately classified subjects to the total number of subjects {(TP+TN)/(TP+TN+FP+FN)}. In addition to accuracy, the specificity and the sensitivity values are also reported. Sensitivity (the proportion of correctly classified positive samples out of all positive returns, or the true positive rate) indicates the accuracy of the prediction group {TP/(TP+FN)}, which in this case is the HC group. Specificity (the proportion of correctly classified negative samples out of all negative returns, or true negative rate), calculated as {TN/(TN+FP)}, indicates the accuracy of the prediction of the absence group, which in this case is the LBP group. To evaluate overall model performance, we performed an area under the ROC (Receiver Operating Characteristics curve) analysis, more commonly referred to as an area under the curve (AUC) analysis.

### I. Statistical Tests

An unpaired two-sample Wilcoxon rank-sum test with p<0.05 was used to evaluate statistically significant differences for group comparisons in both structural and functional data. To correct for multiple comparisons, we used False Discovery Rate Correction (FDR) with q<0.05.

### Results

### A. Clinical Surveys

We compared the LBP SF-36 summary scale scores to HC using a non-parametric Wilcoxon rank-sum test. A Wilcoxon rank-sum test was used to find differences in SF-36-subscores between HC and LBP, with higher scores indicating healthier functioning. There were statistically significant (p<0.05) differences in sub-scores between patient groups except for the RLE sub-score (Table 11). Higher differences were seen in the physical domains (PF, RLP, P, and GH) than in the emotional domain (EF, SF, RLE, EW). These results indicated that LBP leads to greater impairment of physical functioning relative to mental functioning.

TABLE 11
Participant demographic and clinical information

| | Healthy | |
|---|---|---|
| Variable | Controls | LBP |
| Participants (n) | 27 | 24 |
| Sex (M/F) | 15/12 | 9/15 |
| Age (in years) | 46.9 ± 17.3 | 53.5 ± 10.2 |
| SF-36 PF score*** | 53.47 ± 28.5 | 92.3 ± 17.7 |
| SF-36 RLP score*** | 46.87 ± 43.5 | 94.4 ± 14.4 |
| SF-36 P score*** | 48.96 ± 17.22 | 86.57 ± 16.7 |
| SF-36 GH score*** | 58.86 ± 19.22 | 80.56 ± 15.1 |
| SF-36 EF score* | 53.95 ± 19.4 | 66.30 ± 18.2 |
| SF-36 SF score** | 70.83 ± 24.90 | 91.67 ± 14.7 |
| SF-36 RLE score | 83.32 ± 32.6 | 95.05 ± 17.8 |
| SF-36 EW score* | 72.67 ± 17.1 | 82.81 ± 11.4 |

PF = physical functioning, RLP = role limitations due to physical health problems, P = bodily pain, GH= general health, EF = energy and fatigue, SF = social functioning, RLE = role limitations due to emotional problems, EW = emotional well-being. (* = p <0.05, ** = p <0.01, *** = p <0.001; p values have been corrected for multiple comparisons using FDR)

To reduce the dimensionality of the SF-36 data, we calculated factor summary scores (PCS and MCS) for the eight SF-36 subscales. The oblique two-factor solution indicated that physical functioning (PF), role limitations due to physical health problems (RLP), bodily pain (P), general health (GH), and social functioning (SF) loaded heavily on the Physical Component Summary (PCS) factor score whereas energy and fatigue (EF), role limitation due to emotional problem (RLE) and emotional well-being (EW) loaded most heavily on the Mental Component Summary (MCS) scores.

We computed the summary scores for the PCS and MCS scores for each subject to use in further analysis as pain and emotion scores. Multivariate analyses were used to assess the relationship between CT, and PCS and MCS scores separately after correcting for age.

### B. Changes in Cortical Thickness

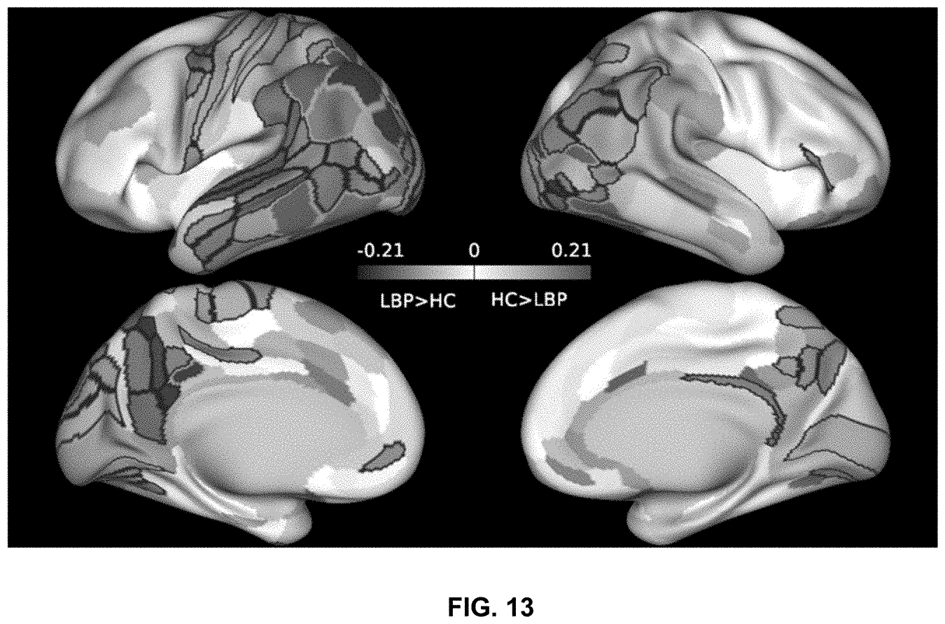

There were widespread differences, both thinning and thickening, in CT between low back pain patients (LBP) and healthy controls (HC). The age-corrected beta parameters for the group differences (the group-as predictor) from the multiple regression analysis were plotted in FIG. 13. The parcels colored in red (HC>LBP) characterized regions having thinner cortexes in LBP relative to HC. The parcels colored in blue (LBP>HC) characterized regions having thicker cortexes in LBP relative to HC. The parcels with a significant group difference (p<0.05, uncorrected) are outlined in black, and parcels that survived multiple comparison corrections (q<0.05) are outlined in green. In general, LBP subjects had widespread regions of thicker cortex within the bilateral occipital, temporal, and parietal lobes. Notably, the posterior cingulate, temporal-parietal junction, and left motor and premotor cortices in both hemispheres showed thicker cortex in LBP patients. These findings were also replicated by a vertex-wise analysis of CT.

### C. Association Between Cortical Thickness and Clinical Summary Scores

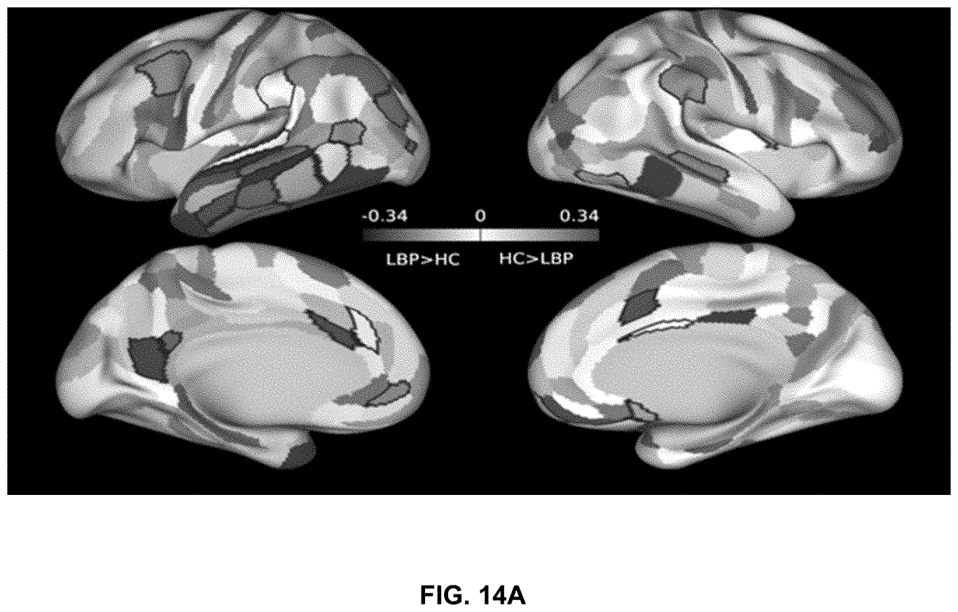

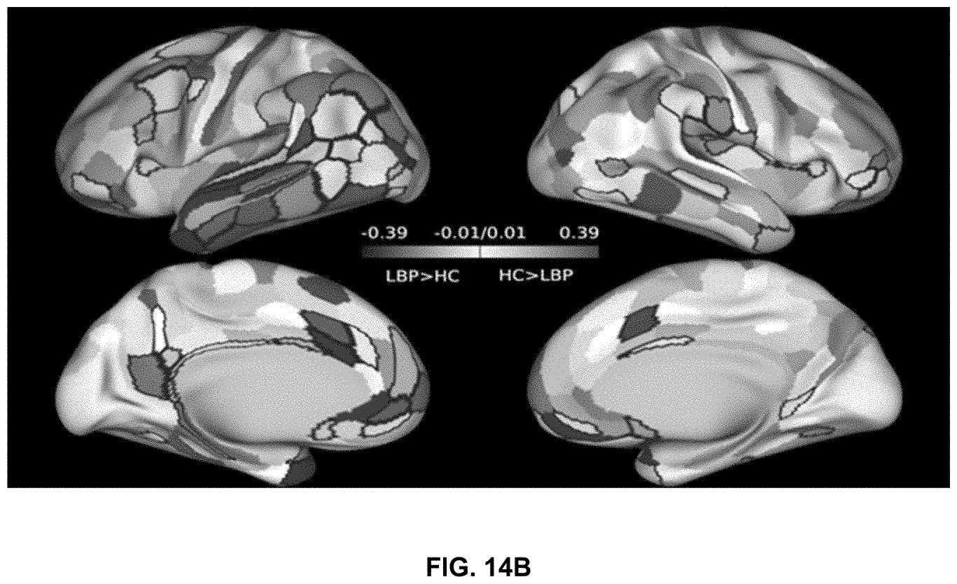

We tested the relationship between the PCS and MCS scores with CT using a linear regression model while controlling for age. Both clinical summary factors were independently found to be significant predictors of the CT of multiple cortical areas (FIGS. 14A and 14B, p<0.05, age-controlled). There were widespread associations that were neither limited to specific functional networks nor specific cortical locations. We also tested the relationship of the PCS and MCS scores with CT within the LBP group.

A higher score for either the PCS or MCS suggests healthier functioning. A negative beta value from the regression (FIG. 14A and FIG. 14B, blue regions) represents regions that show a positive association between the summary score and CT in LBP. Similarly, a positive beta value (red regions) represents regions that show a negative association between the respective summary score and CT in LBP.

D. Parcel and Network rsFC Analysis

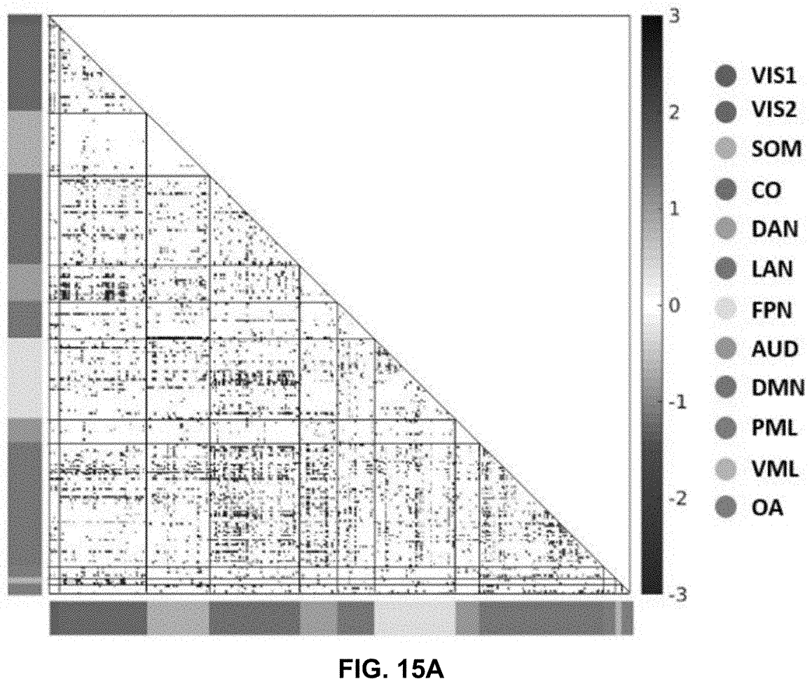

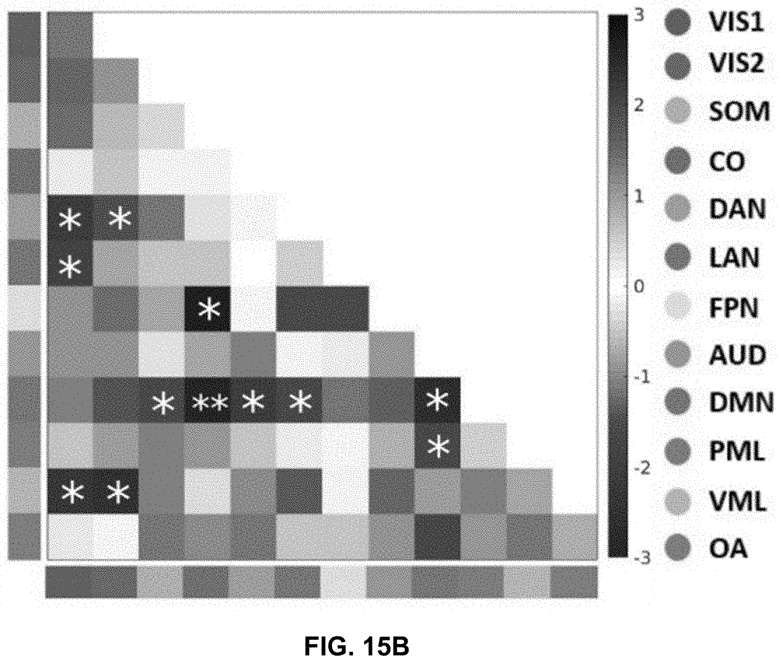

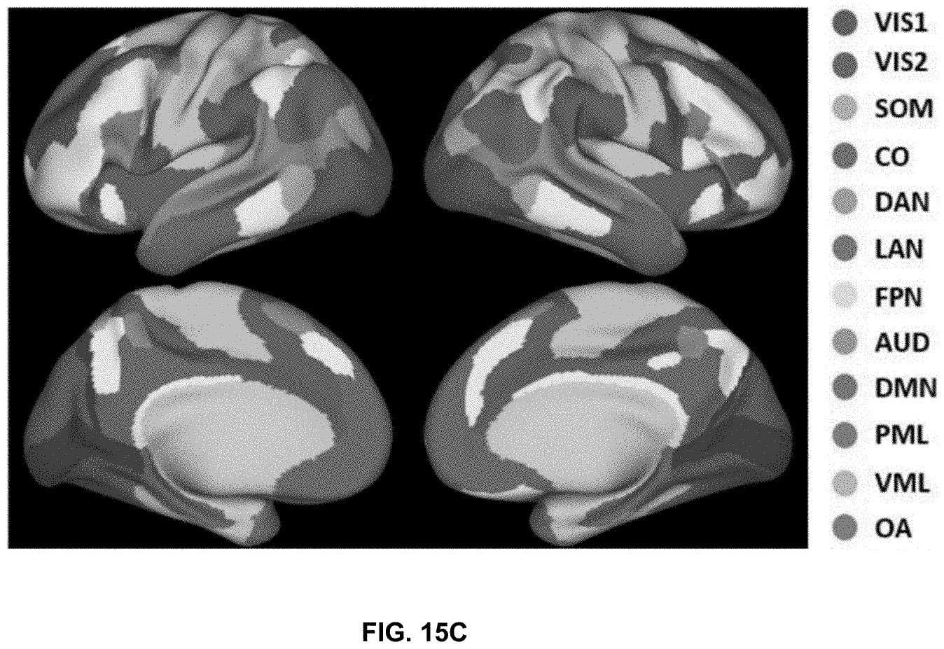

Differences in rsFC between LBP and HC were calculated as described above. FIG. 15A shows the parcels reordered by a network that showed significant (p<0.05 uncorrected) differences in connectivity. We also computed group differences of inter-network and intra-network functional connectivity. There were multiple statistically significant differences in inter-network connectivity interactions as shown in FIG. 15B. We determined that age was not a significant predictor of the network functional connectivity interactions (shown in FIG. 15B) using linear regression analysis. FIG. 15C shows the resting state networks plotted on the cortical surface as outlined by the Cole-Anticevic Brain Network Parcellation.

### E. LBP and HC had Similar Global Graph Metrics

There were no significant differences in global efficiency or clustering coefficient, of the reconstructed brain networks between LBP patients and HC. However, there was a significant difference in the characteristic path length of the reconstructed brain networks between LBP patients and HC (z=2.236, p=0.0253). Global efficiency places a smaller influence on parcels that are isolated from the network when compared to characteristic path length. Since we didn't observe a significant difference in the global efficiency between both patient cohorts, we can conclude that the reconstructed brain networks of LBP patients had more isolated parcels than HC.

### F. Changes in Network Efficiency

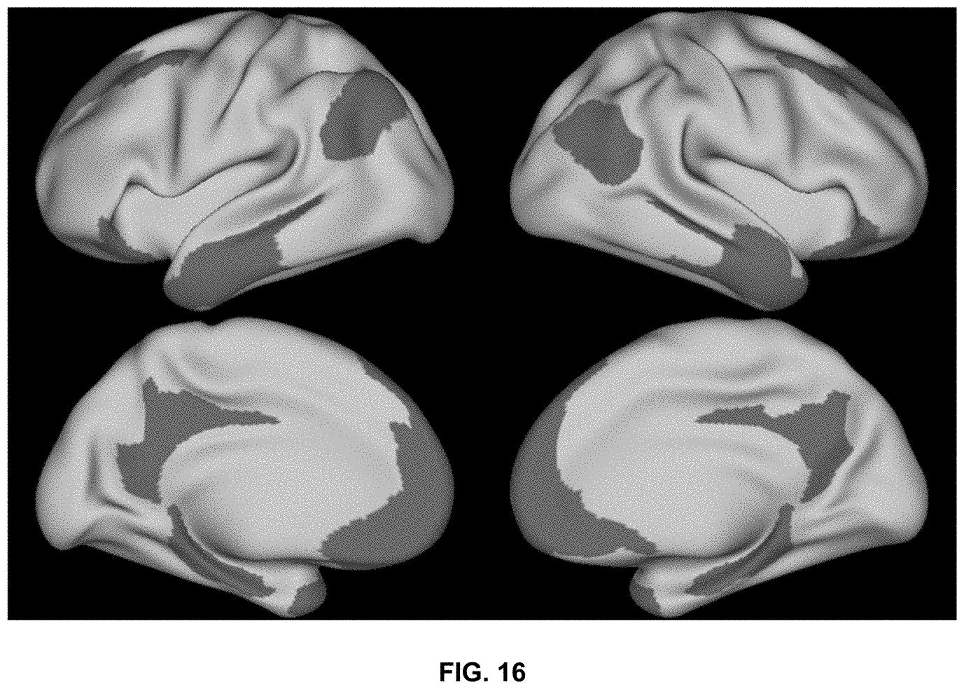

We next investigated changes in network efficiency within each of the 12 resting-state networks in LBP when compared with HC. There was a statistically significant decrease (z=−2.10, p=0.0320 uncorrected) in the network efficiency of the default mode network (see the cortical map of DMN in FIG. 16) in LBP and trending significant differences in the frontoparietal and ventral multimodal networks as shown in Table 12. rsFC data was used to compile binary undirected networks for each resting-state network that had been thresholded within a network density range of 15%-30% in steps of 2.5%. The corresponding global efficiency scores for each network rsFC matrix were averaged across thresholds. A Wilcoxon rank-sum test was used to assess the statistical significance and the corresponding z values recorded. (*=p<0.05, uncorrected).

TABLE 12
Global efficiency of network in LBP.

| Network Names | Z-Value | p-Value |
|---|---|---|
| Primary Visual | 1.60 | 0.110 |
| Secondary Visual | 1.30 | 0.180 |
| Somatomotor | 0.0850 | 0.930 |
| Cingulo-Opercular | −0.580 | 0.560 |
| Dorsal Attention | 0.410 | 0.680 |
| Language | 0.330 | 0.750 |
| Frontoparietal | −1.80 | 0.0690 |
| Auditory | −0.590 | 0.550 |
| Default Mode | −2.10 | 0.0320* |
| Posterior-Multimodal | 1.20 | 0.230 |
| Ventral-Multimodal | 1.90 | 0.0640 |
| Orbito-Affective | −0.12 | 0.910 |

### G. Nature of Brain's Hub Structure in LBP

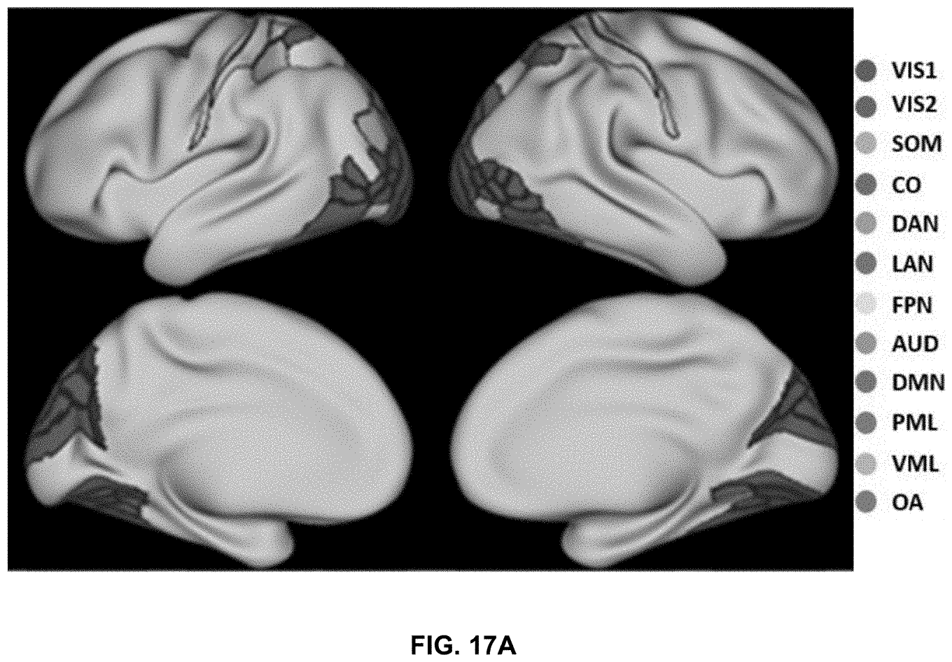

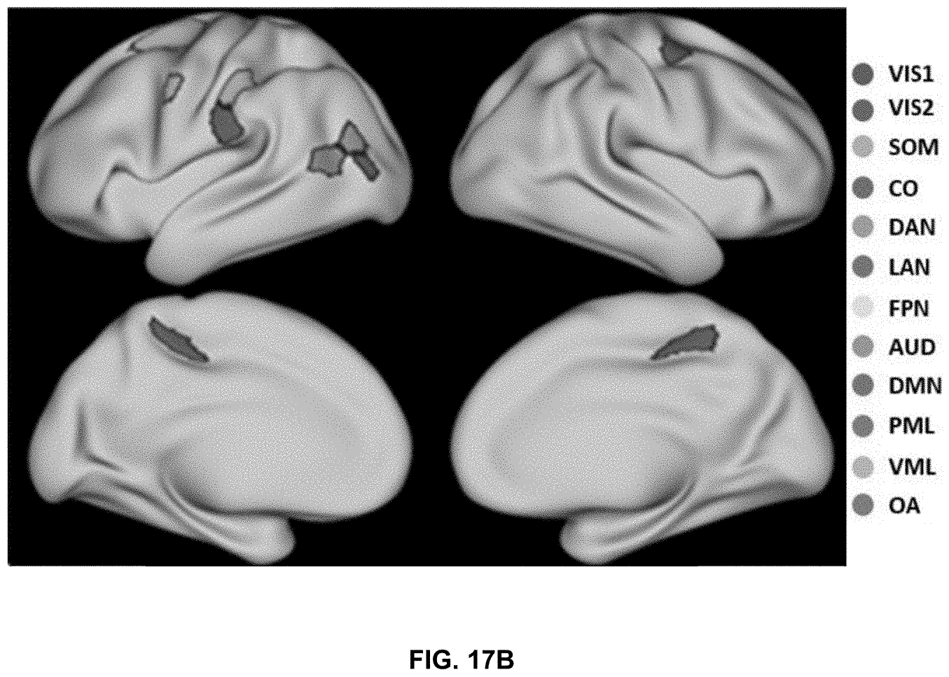

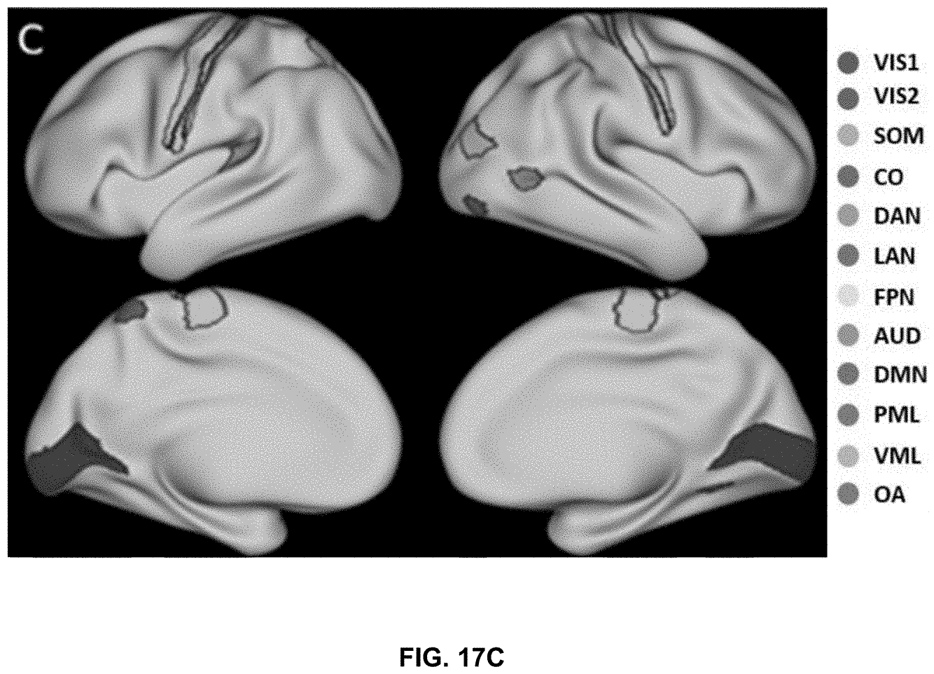

We calculated the eigenvector centrality of each node to investigate the nature of its connections with surrounding nodes. A hub was defined as a node whose eigenvector centrality was one standard deviation above the group mean. As a result, we identified hubs that were found in 1) both LBP and HC, 2) HC but not in LBP, and 3) LBP but not in HC, and then matched each of the corresponding hubs to their respective resting-state networks. The hubs for each of the three conditions were then projected onto a brain mesh surface (shown in FIGS. 17A, 17B, and 17C, respectively).

### H. Machine Learning Classification of LBP and HC Groups

We used the cortical thickness (CT) as the feature to train a support vector machine to accurately classify each subject to their respective patient group as described above. Table 13 summarizes the overall classification results. When classifying LBP from HC, we achieved a classification accuracy of 74.51%, AUC of 0.787 (95% CI: 0.66-0.91), a sensitivity of 74.07%, and a specificity of 75.00%.

TABLE 13
A summary of the classification accuracy and AUC
when cortical thickness was used to train the SVM model.

| SVM (LOO) | Accuracy | Sensitivity | Specificity | AUC |
|---|---|---|---|---|
| LBP vs HC | 74.51% | 74.07% | 75.00% | 0.787 |

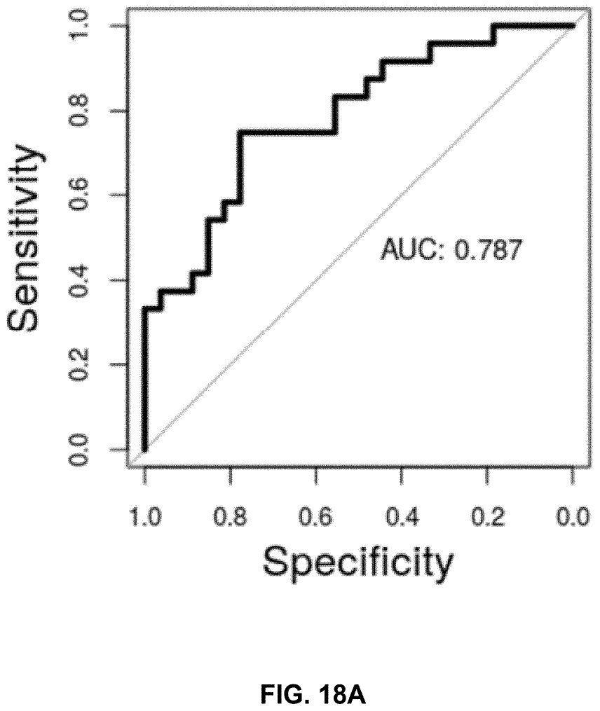

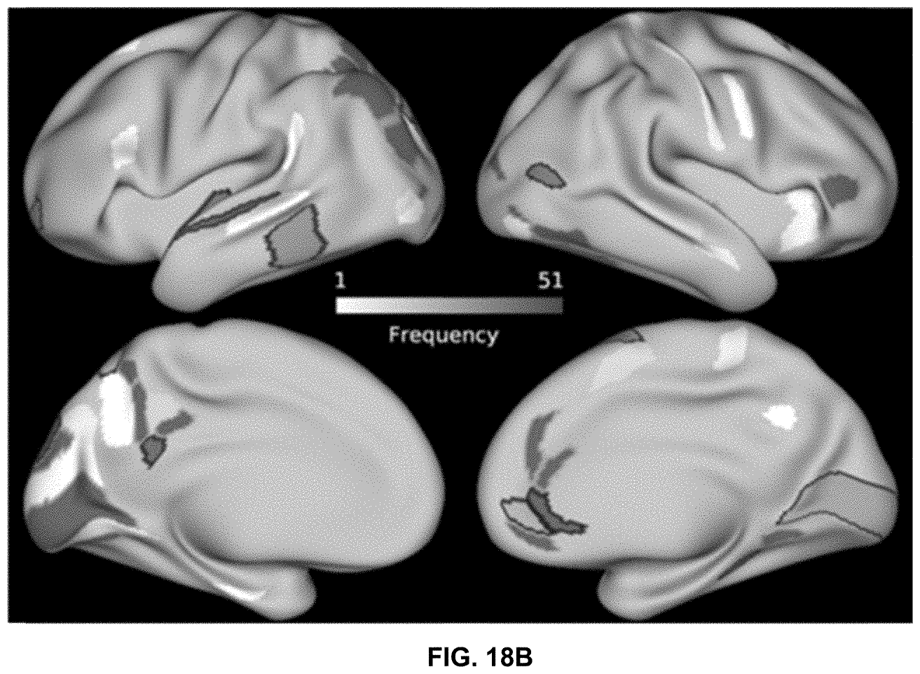

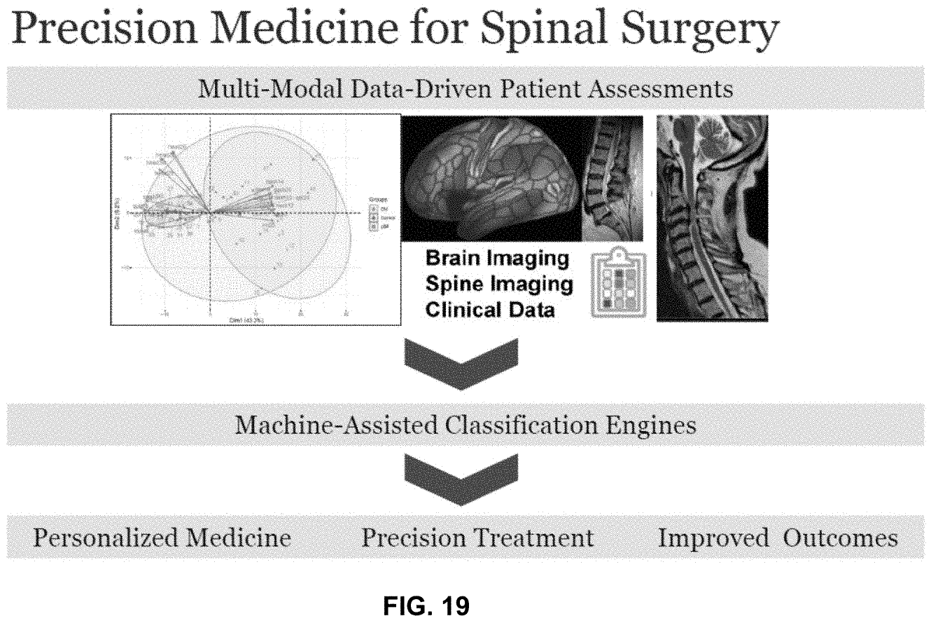

The receiver operating characteristic (ROC) curves for stratifying patients is shown in FIG. 18A. The cortical areas contributing to the classification and their corresponding frequency values, quantified number of repetitions out of 51 LOO iterations, were plotted on a brain mesh surface (FIG. 18B).

### Discussion

In this study, we identified structural and functional biomarkers in LBP patients by applying a multi-modal approach using a surface-based cortical parcellation. The results revealed the following in LBP patients: 1) Differences in CT between LBP and HC, 2) associations between CT and self-reported clinical scores, 3) decreased functional connectivity between multiple networks, 4) lower network efficiency of the default mode network, and 5) changes to hub topology of the brain. In addition, a support vector machine trained using CT values achieved a very high level of accuracy differentiating LBP from HC.

### A. Cortical Thickness as a Predictor of Pain and Emotion Scores

Several studies have observed grey matter decreases with longer pain duration in the dorsolateral prefrontal cortex, insular cortex, and anterior and dorsal anterior cingulate cortices. These areas have been described as vulnerable due to stress, which may indicate that gray matter decreases are a consequence of chronic pain and anxiety that is not unique to LBP. In our study, decreases in the CT of these regions were not statistically significant in our LBP population. As reported in previous studies, there were significant increases in CT of the posterior parietal junction, temporal-parietal junction, and visual-processing stream (FDR corrected p<0.05, FIG. 13) in our LBP cohort. In addition, the cortical areas contributing to the classification of patients using CT (FIG. 18B) were consistent with previously published findings in chronic LBP patients. These included regions such as the temporal, sensory-motor, cingulate, and prefrontal cortices which are commonly implicated in pain processing.

We also tested the relationship between the degree of pain and emotion with CT. The CT in the left dorsolateral prefrontal cortex, anterior cingulate cortex, midcingulate cortex, posterior cingulate cortex, posterior parietal cortex, and lateral temporal cortices predicted clinical pain scores. LBP patients commonly exhibit emotional and cognitive disorders, including depression, anxiety, and sleep disturbances. Appropriately, the parcels which predicted the subject-reported pain scores are known to be involved in the limbic processing of emotion and affective control in LBP patients.

Many parcels included significant correlations with both pain and emotion summary scores. The effects of pain and emotion are known to coexist in LBP and thus this overlap was expected to be seen in the neuronal circuitry of the brain. However, it is not known whether this overlap in pain and emotional scores reflects a common underlying pathophysiological process or a mutually exclusive process. Few studies have documented increases in gray matter volume in the premotor cortex, midcingulate cortex, Si, inferior parietal lobule, and the medial temporal gyrus in the presence of pain stimuli. Regions within the temporal lobe, including the medial and inferior temporal gyrus are associated with pain and emotion in studies using different paradigms, such as during emotion anticipation and facial expression of pain. Based on our findings, we believe these regions may also be involved in the affective component of LBP.

### B. Visual Network Plasticity During LBP

In humans, spatial navigation is a complex process that involves the processing of multiple incoming sensory stimuli based on surrounding spatial landmarks to determine the optimal route to a specific goal. In a recent systematic review, one factor common to all chronic LBP patients was impaired proprioception. Impaired proprioception was also far worse in patients with severe chronic LBP. Proprioception is an important sensory input that functions to provide the perception of the body (i.e. physical self-awareness) and judgment of alignment relative to one's environment. Due to impaired cortical processing of proprioceptive input, patients with chronic LBP exhibit aberrant perception, and consequently alignment of their bodies relative to their surroundings.

To compensate for proprioception impairment, vision becomes the next reliable sensory feedback that helps in spatial orientation, movement coordination, and balance. In patients with chronic LBP, several studies have demonstrated that dependence on visual input increases in order to maintain a vertical posture. When visual input is removed or reduced, patients with chronic LBP have increased postural sway and loss of balance. These studies support the visual dependence in patients with chronic LBP. Within our LBP cohort, we found multiple parcels from the visual networks were highly predictive of LBP when using a classification algorithm trained using cortical thickness (FIG. 18B). We also found multiple increases in connectivity between the visual networks and other RSNs. These increases in connectivity could be a result of the visual system prioritizing tasks such as maintaining verticality and posture while placing less emphasis on the control of attentional tasks. The presence of the primary visual cortex as a hub in LBP patients and not in HC is essential in coordinating this increase in network processing and information exchange to aid in proprioception.

### C. Role of the DMN Network in LBP

Chronic pain is an attention-demanding process, often competing with other external stimuli for cognitive resources. Individuals across many chronic pain states show deficits in attention. The default mode network (DMN) is composed of many higher-order cognitive processing regions including the medial prefrontal cortex, posterior cingulate cortex, inferior parietal cortex, and precuneus. While it is still unclear what the DMN is responsible for, elements of its networks have been implicated in episodic memory, modulation of pain perception, and monitoring the external environment. There have been many recent studies that support the reorganization of DMN function across many chronic pain states.

In this study, several parcels from the DMN were highly predictive of LBP when using a classification algorithm trained using cortical thickness (FIG. 18B). The DMN also showed increased (both significant and non-significant) connectivity with most other RSNs in LBP patients. However, the DMN showed a significant decrease in connectivity with nodes within its network in LBP patients. In addition, there was a significant decrease in the network efficiency of the DMN. Executive functions are laborsome requiring the availability of resources which is achieved by reducing the activation of the DMN. A decrease in the efficiency of the DMN in LBP patients might affect the induced deactivation of this network and hence compromise their executive functions. Recent data from patients with Alzheimer's disease and attention-deficit/hyperactivity disorder show the role of the DMN in executive function deficit. This decrease in network efficiency explains the hyperactive connectivity we observe between the DMN and all other RSNs in LBP patients.

### D. Hub Reorganization in Sensorimotor Processing

Of primary importance is the role of the bilateral primary motor cortex in regulating the flow of information specifically by acting as a hub within LBP patients but not HC. The motor cortex has been implicated in a number of functions beyond motor control such as visuomotor transformations, language processing, memory retrieval, and pain processing. It has been proposed that incongruence between motor intention and movement, or sensorimotor conflict, is responsible for increased activation of Ml. Systems responsible for motor function are closely linked to sensory feedback systems, which are monitored to detect deviations from the predicted response. In HC, presenting conflicting information, such as a mismatch between intention, proprioception, or visual feedback induced pain and sensory disturbances and aggravated symptoms in those with chronic pain.

Patients with chronic LBP frequently experience proprioception deficits and tactile acuity deficits. A hyper-efficient posterior multimodal network combined with the abnormal proprioceptive representation of the low back in the primary somatosensory cortex may contribute to sensorimotor conflicts in patients with chronic LBP. The lack of visual input of moving segments and reduced activity in vision processing centers can enhance sensorimotor conflicts, as vision is the dominant form of perception. In addition, the lack of visual feedback means that atypical cortical proprioceptive representation cannot be corrected. These alterations in proprioceptive representation, visual perception, and sensorimotor conflicts lead to downstream effects in higher-order pain processing centers which may directly produce pain and sustain altered motor control strategies.

### E. SVM Classifier Trained Using Cortical Thickness

A clinically usable finding in this study is the development of a machine learning classification engine that can predict patient groups based on differences in cortical thickness. Recent studies have attempted to predict patient groups in chronic pain states using structural features. However, this is the first study to demonstrate the advantage of using structural features derived from brain imaging parcellated using an MMP when discerning between LBP and HC patient groups. We trained the classifier using CT which achieved a maximum classification accuracy of 74.51% (AUC=0.787, 95% CI: 0.66-0.91). The results validated our hypothesis that widespread changes in CT can be used as an imaging biomarker for LBP to guide therapy.

### Conclusion

The results of these experiments included the observation of widespread differences in cortical thickness (CT) in patients with low back pain relative to healthy controls. These observed changes in CT were correlated with self-reported clinical scores of pain and emotion. In addition, changes in the resting-state fMRI metrics of functional networks were observed. Support vector machines were able to separate low back pain patients from healthy controls with a very high level of classification performance. The results of these experiments identified multi-modal biomarkers as potentially useful for identifying personalized treatments for low back pain.

The results of these experiments suggest that low back pain is associated with widespread structural and functional changes in the brain. Our data shows that localized structural changes are correlated with clinical pain and emotional measures. The resting-state functional connectivity and graph theory network approaches further support the findings of alterations of brain structure and functions localized to regions corresponding to cognitive functions, visuo-motor, and affective dimensions of pain processing. The results of these experiments also demonstrate how machine-assisted classification algorithms can accurately categorize patient-specific data into their respective cohort using data derived from a multi-modal parcellation.

### Example 3: Identification of Biomarkers for Low Back Pain (LBP)

To assess the ability of multimodal biomarkers to predict clinical metrics, the following experiments were conducted. A population of patients diagnosed with back pain disorders, including Severe Myelopathy (mJOA<12), Traumatic Spinal Cord Injury, or Severe Back Pain (Disability index>50) were selected. The patient population included 15 patients with spinal cord compression/myelopathy, 24 patients with chronic back pain, and 27 age-matched controls. Serial assessments were available for 7 of the subjects (2 myelopathies, 5 LBP). Assessments were also obtained for chronic back pain subjects before and after treatment. The assessments included MRI imaging and various clinical phenotyping domains that included approximately patient-reported outcomes measures. Table 14 lists various clinical phenotyping domains, representing over 200 clinical data points that were collected for analysis as described below.

TABLE 14
Clinical Phenotyping Domains.
Clinician-Measured Data

| | |
|---|---|
| Medical Histories | Complete neurological examinations |

Patient-Reported Outcome Scores

| | |
|---|---|
| Int. Standards for Neuro. Validated | Disabilities of Arm, |
| Patient-Reported Outcome | Shoulder and Hand |
| PROMIS-29 | Scoliosis Society Questionnaire |
| Oswestry Disability Index | Rolland Morris Pain Questionnaire |
| SF-36 | Neck Disability |
| Mod. Jap. Ortho. Association Score | McGill Pain Questionnaire |
| Shoulder Pain Score | Carpal tunnel Questionnaire |
| Quadruple Visual Analog Pain scale | Headache Disability Index |

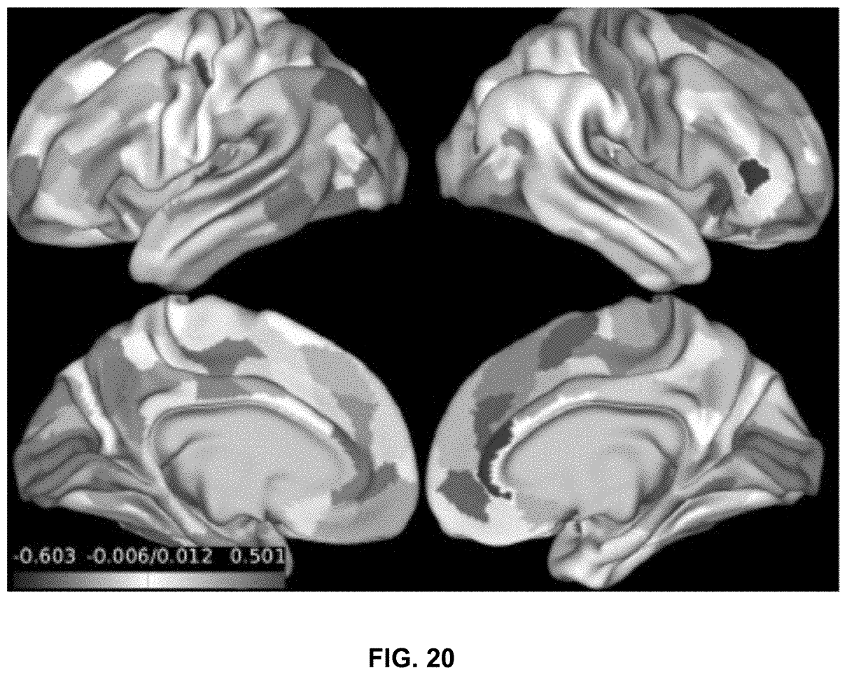

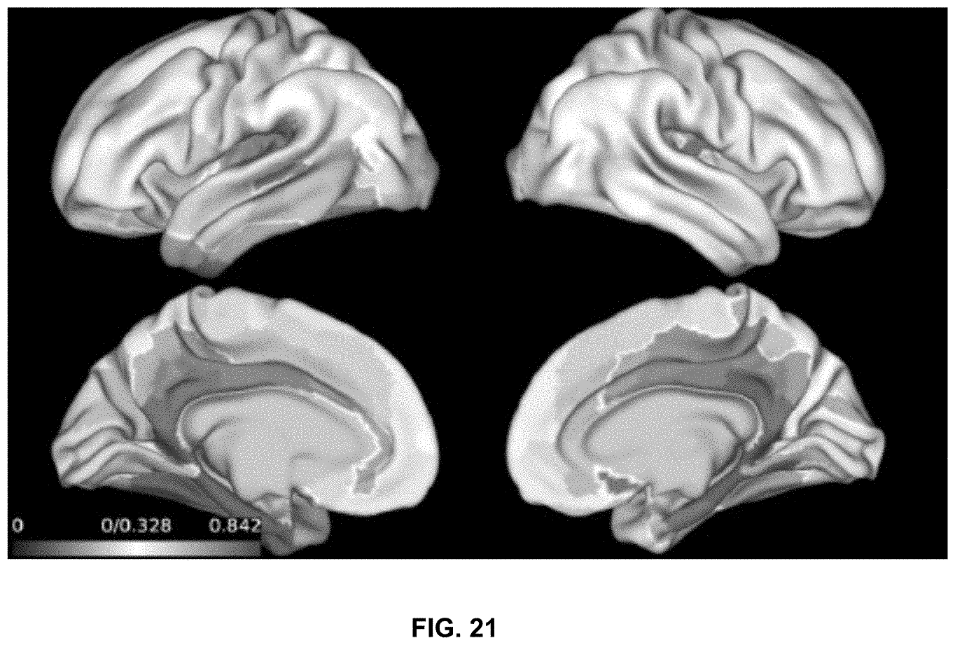

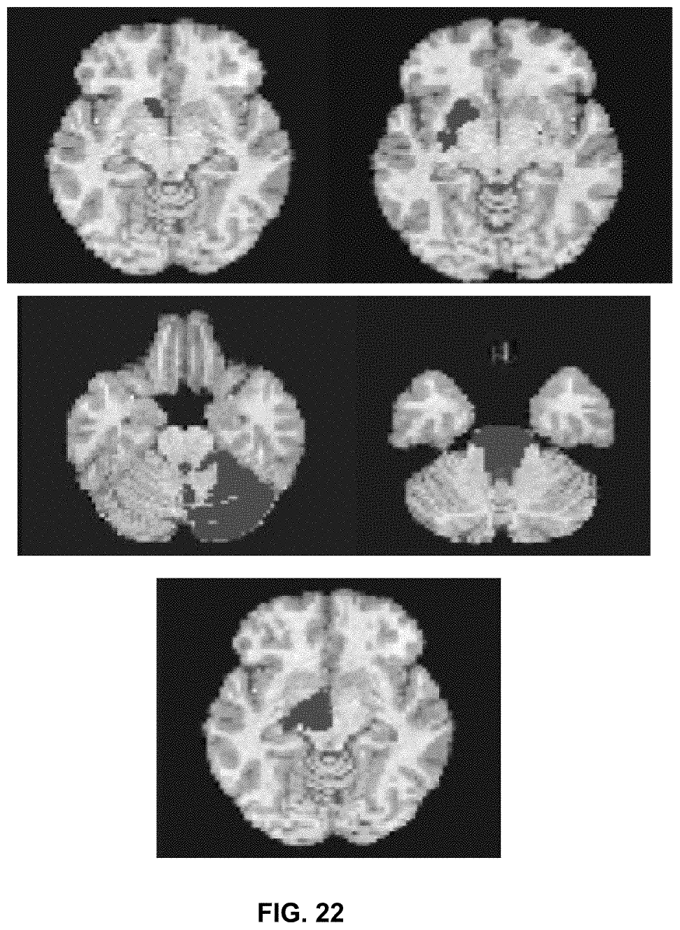

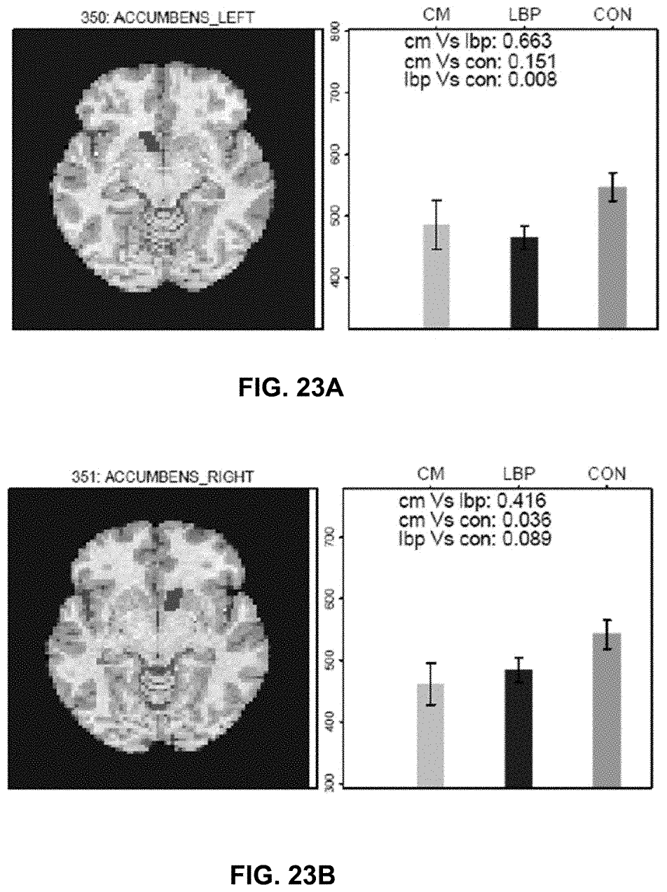

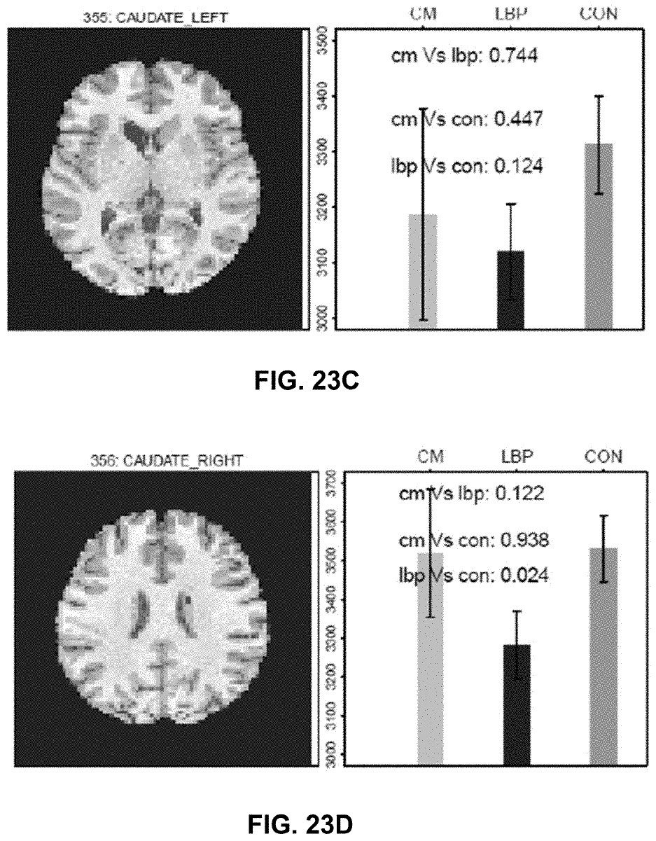

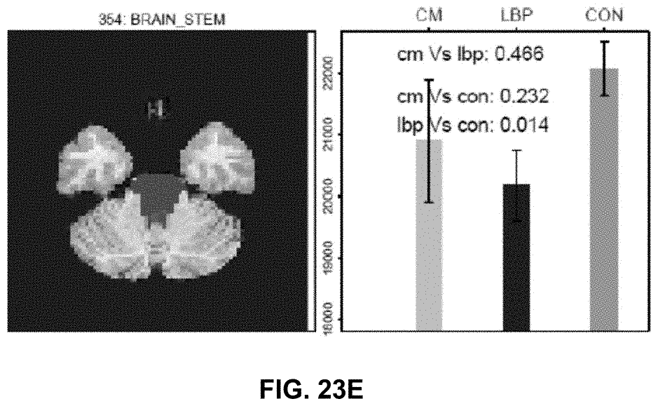

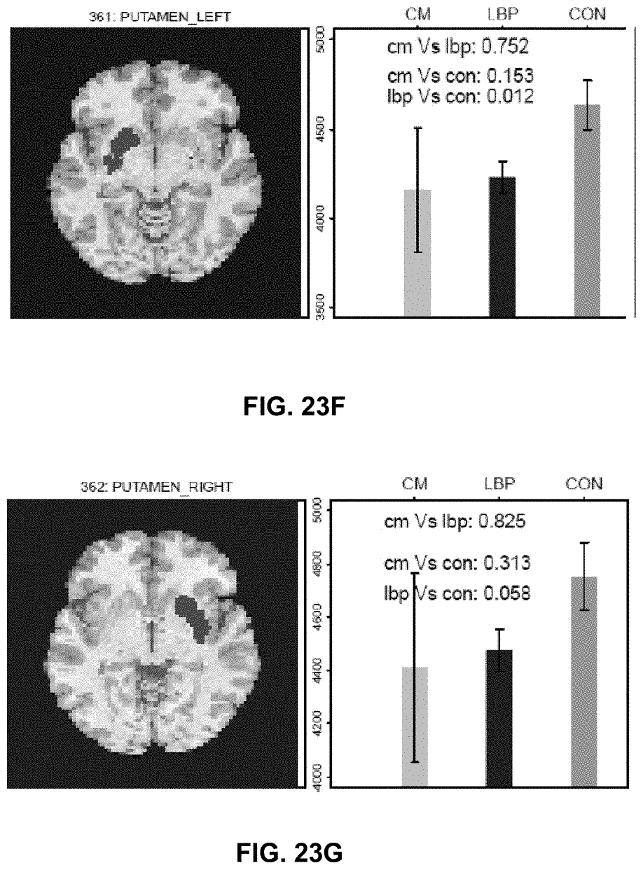

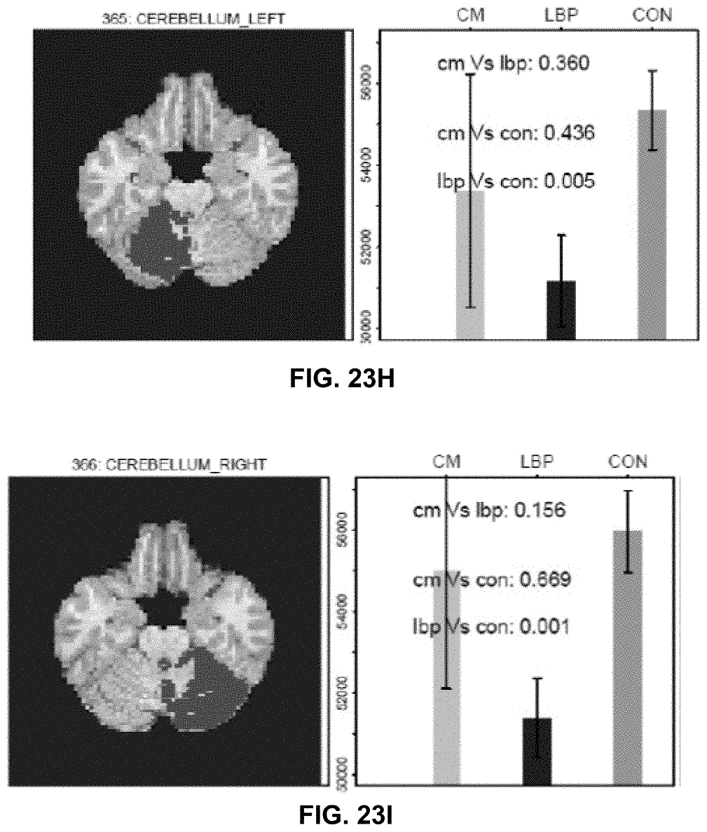

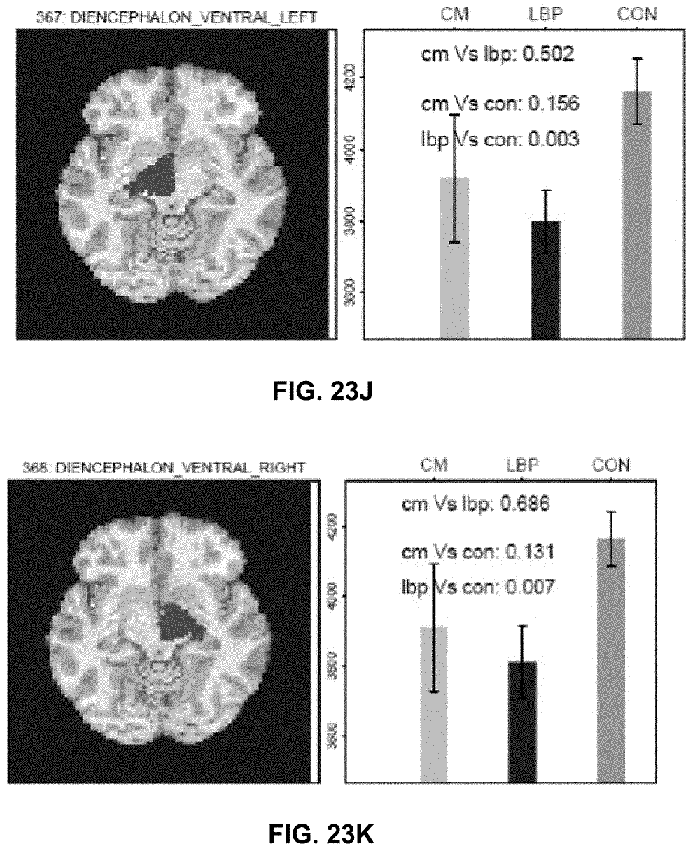

Structural MRI data obtained from a total of 48 subjects and controls were analyzed as described above to obtain spatial distributions of cortical thickness, myelin content, and grey matter volume. Pooled data for the chronic back pain subjects were compared to the pooled data for the controls as described above. FIGS. 20, 21, and 22 are comparisons of the cortical thickness, myelin content, and grey matter volume distributions, respectively, of the control group with the chronic back pain patients. The subcortical grey matter volumes of CM (cervical myelopathy), LBP (low back pain), and healthy control (CON) groups were compared within various brain regions, as shown in FIGS. 23A, 23B, 23C, 23D, 23E, 23F, 23G, 23H, 23I, 23J, and 23K.

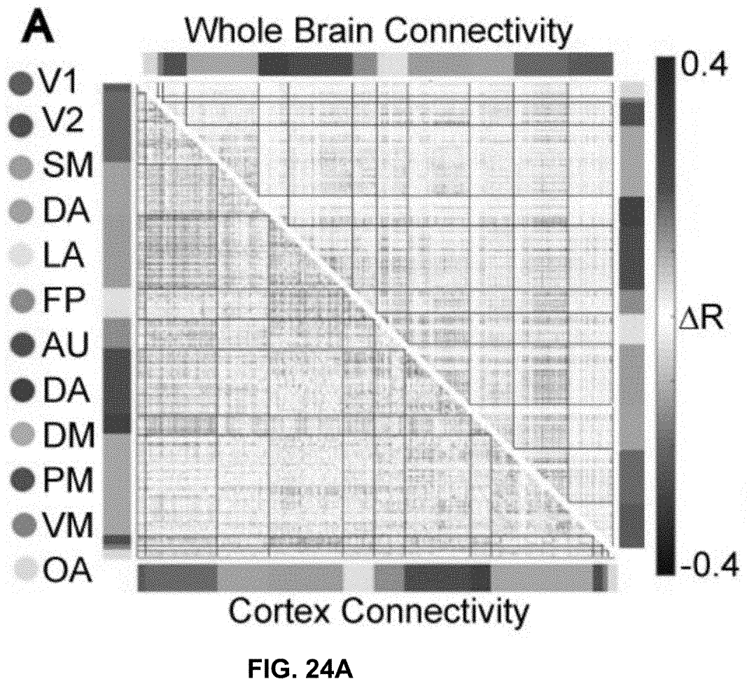

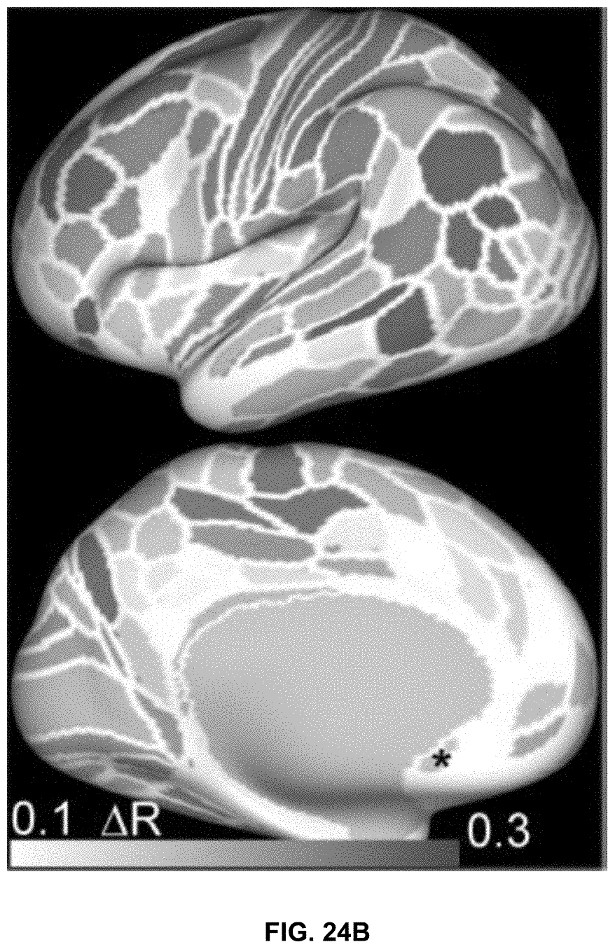

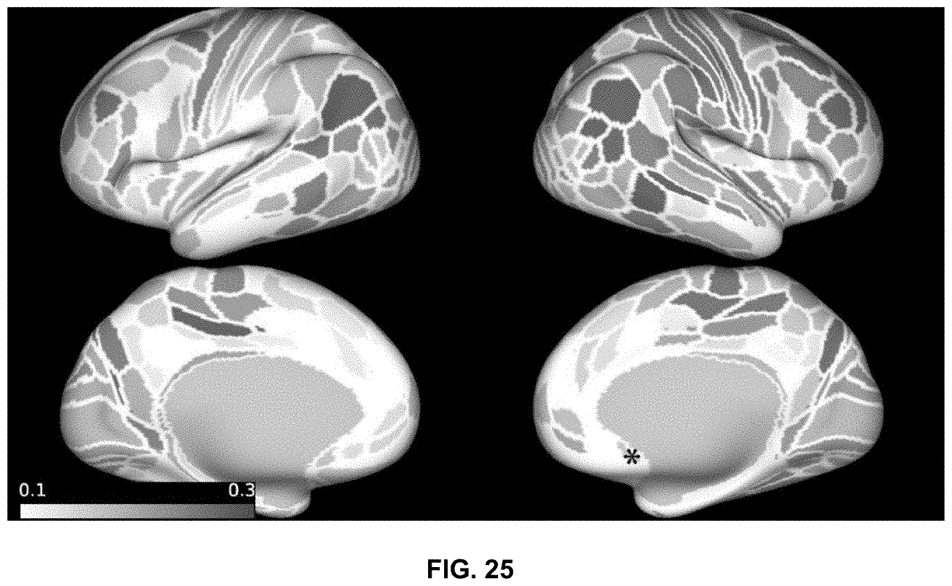

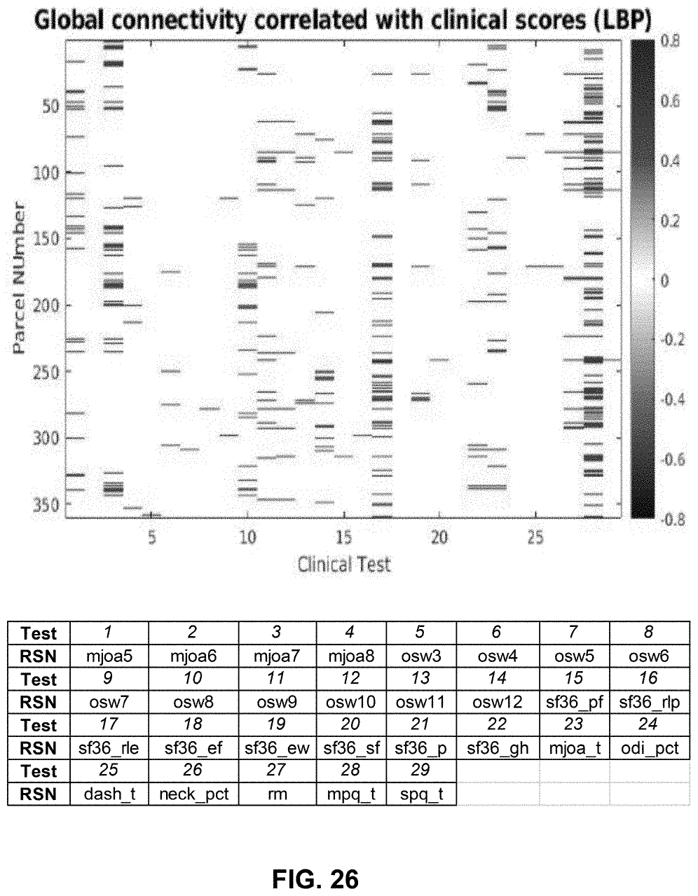

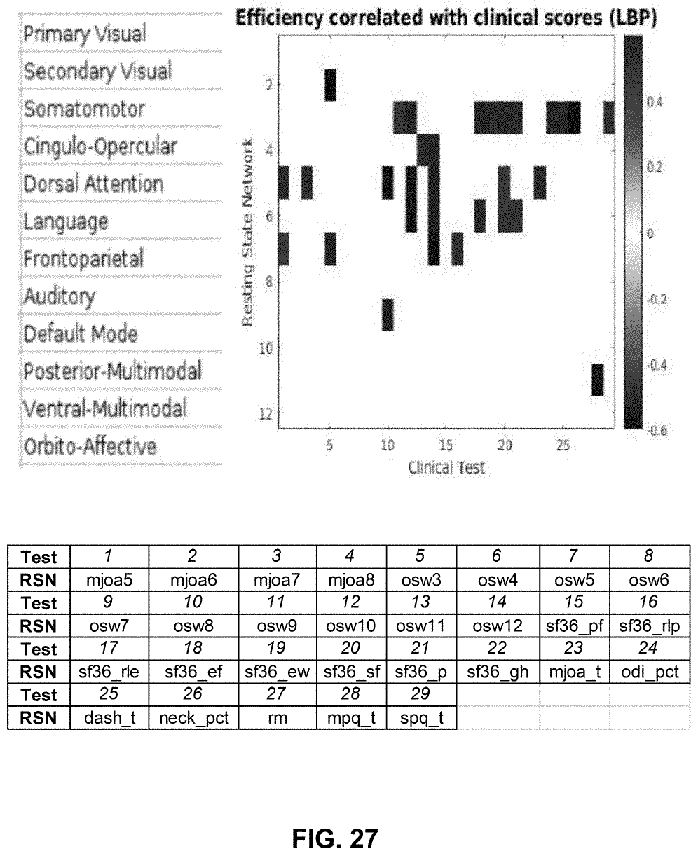

Whole-brain and cortex connectivity of the various resting-state networks were also assessed within the pooled chronic back pain (CPB) and control (CON) groups as described above. Changes in whole brain and cortex connectivity of the various resting-state networks in CPB versus CON groups are summarized on the above-diagonal and below-diagonal regions of FIG. 24A. Red denotes changes in connectivities where CPB<CON and blue denotes changes in connectivities where CPB>CON. FIG. 24B is a spatial map of the changes in connectivity summarized in FIG. 24A. FIG. 25 is a cortical map of changes in global connectivity to the subgenual cingulate gyrus. A linear regression analysis was performed to identify correlations between the changes in global connectivity and a subset of the clinical scores of Table 14. FIG. 26 is a graph summarizing the correlations of global connectivity changes with various clinical scores. FIG. 27 is a graph summarizing the correlations of function network efficiencies with various clinical scores.

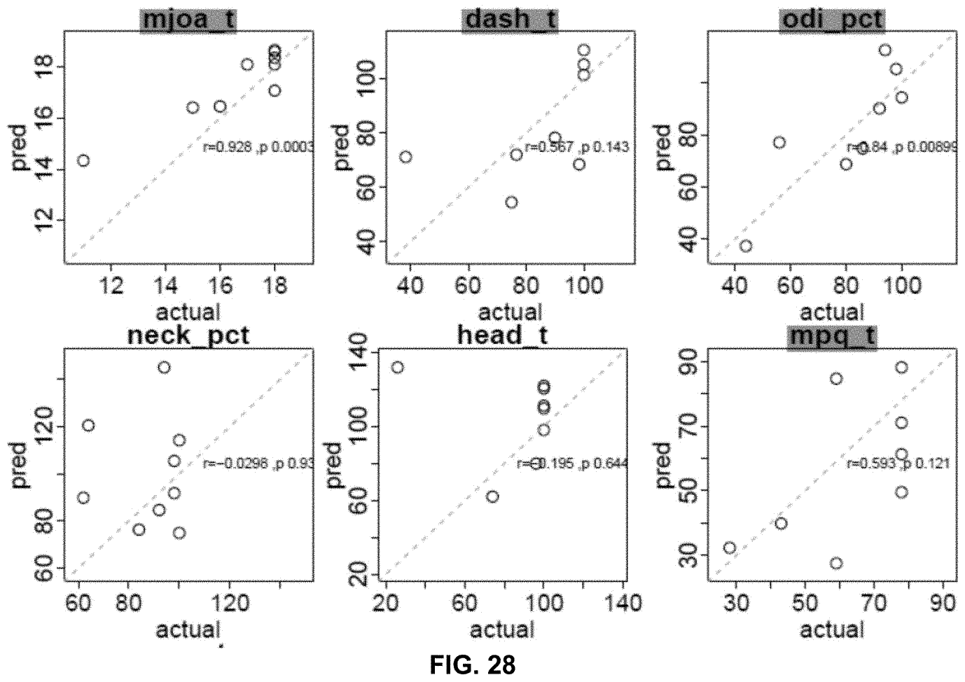

A support vector machine (SVM) was trained as described above using portions of the structural and functional MRI measurements described above indexed to various patient-reported outcome scores of Table 14. A first SVM model was trained as described above using cortical thickness only, and a second SVM model was trained using cortical thickness, subcortical volume, and global connectivity. Both SVM models were able to predict whether a person has back pain with at least 95% accuracy. In addition, the second SVM model based on cortical thickness, subcortical volume, and global connectivity was able to predict a variety of patient self-reported clinical scores, as illustrated in FIG. 28.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

What is claimed is:

1. A multi-modal biomarker predictive of a pain level in a patient, the biomarker comprising at least one of a structural MRI-based parameter from a brain of the patient and a functional MRI-based parameter from the brain of the patient.

2. The biomarker of claim 1, wherein the structural MRI-based parameter comprises at least one of a cortical thickness and a sub-cortical volume and the functional MRI-based parameter comprises at least one of a resting-state functional connectivity matrix parameter and a graph metric parameter, the graph parameter comprising a global efficiency, a clustering coefficient, and a characteristic path length.

3. The biomarker of claim 2, wherein the resting-state functional connectivity matrix parameter comprises a global connectivity.

4. The biomarker of claim 3, the biomarker consisting of the cortical thickness, the sub-cortical volume, and the global connectivity.

5. A computer-implemented method of estimating a pain level in a patient based on a multi-modal biomarker, the method comprising:
a. providing to a computing device the multi-modal biomarker comprising at least one of a structural MRI-based parameter from a brain of the patient and a functional MRI-based parameter from the brain of the patient; and
b. transforming, using the computing device, the multi-modal biomarker into the estimated pain level using a machine learning model.

6. The method of claim 5, wherein the machine learning model comprises a support vector machine.

7. The method of claim 6, wherein the structural MRI-based parameter comprises at least one of a cortical thickness and a sub-cortical volume and the functional MRI-based parameter comprises at least one of a resting-state functional connectivity matrix parameter and a graph metric parameter, the graph metric parameter comprising at least one of a global efficiency, a clustering coefficient, and a characteristic path length.

8. The method of claim 7, wherein the resting-state functional connectivity matrix parameter comprises global connectivity.

9. The method of claim 8, wherein the multi-modal biomarker consists of the cortical thickness, the sub-cortical volume, and the global connectivity.

10. The method of claim 9, wherein the estimated pain level comprises an estimated clinical score comprising a score from at least one of a Modified Japanese Orthopedic Association, a Oswestry Disability Index, an SF-36, a Disabilities of Arm, Shoulder and Hand, a Neck Disability, a Rolland Morris Pain Questionnaire, a McGill Pain Questionnaire, a Shoulder Pain Score, any portion thereof, and any combination thereof.

11. The method of claim 10, further comprising training, using the computing device, the machine learning model using a training dataset, the training dataset comprising a plurality of entries, each entry comprising a multimodal biomarker and an associated clinical score for a training patient from a population of pain patients.

12. A system for estimating a pain level in a patient based on a multi-modal biomarker, the system comprising a computing device comprising at least one processor and a non-volatile computer-readable media, the non-volatile computer-readable media containing instructions executable on the at least one processor to transform the multi-modal biomarker into the estimated pain level using a machine learning model.

13. The system of claim 12, wherein the machine learning model comprises a support vector machine.

14. The system of claim 13, wherein the structural MRI-based parameter comprises at least one of a cortical thickness and a sub-cortical volume, and the functional MRI-based parameter comprises at least one of a resting-state functional connectivity matrix parameter and a graph metric parameter, the graph metric parameter comprising at least one of a global efficiency, a clustering coefficient, and a characteristic path length.

15. The system of claim 14, wherein the resting-state functional connectivity matrix parameter comprises a global connectivity.

16. The system of claim 15, wherein the multi-modal biomarker consists of the cortical thickness, the sub-cortical volume, and the global connectivity.

17. The system of claim 16, wherein the estimated pain level comprises an estimated clinical score comprising a score from at least one of a Modified Japanese Orthopedic Association, a Oswestry Disability Index, an SF-36, a Disabilities of Arm, Shoulder and Hand, a Neck Disability, a Rolland Morris Pain Questionnaire, a McGill Pain Questionnaire, a Shoulder Pain Score, any portion thereof, and any combination thereof.

18. The system of claim 17, wherein the non-volatile computer-readable media further contains instructions executable on the at least one processor to train the machine learning model using a training dataset, the training dataset comprising a plurality of entries, each entry comprising a multimodal biomarker and an associated clinical score for a training patient from a population of pain patients.
